(19) United States
(12) Patent Application Publication
(10) Pub. No.: US 2026/0058007 A1
(43) Pub. Date: Feb. 26, 2026

(54) REAL-TIME ARTIFACT PROCESSING AND FEATURE EXTRACTION METHOD AND SYSTEM FOR ELECTROENCEPHALOGRAM SIGNAL

(71) Applicants: KINGFAR INTERNATIONAL INC., Beijing (CN); Nanjing Kingfar Health Technology Inc., Nanjing (CN)

(72) Inventors: Qichao ZHAO, Beijing (CN); Ran YANG, Beijing (CN); Qingju WANG, Beijing (CN)

(21) Appl. No.: 19/379,681

(22) Filed: Nov. 4, 2025

Related U.S. Application Data
(63) Continuation of application No. PCT/CN2024/140724, filed on Dec. 19, 2024.

(30) Foreign Application Priority Data
Dec. 21, 2023 (CN) 202311775102.6
Dec. 27, 2023 (CN) 202311823752.3
Dec. 28, 2023 (CN) 202311844300.3

(51) Int. Cl. G16H 40/60; A61B 5/00; A61B 5/31; A61B 5/374

(52) U.S. Cl. CPC G16H 40/60; A61B 5/31; A61B 5/374; A61B 5/7267

(57) ABSTRACT

Provided are a method and a system for real-time artifact processing and feature extraction of an electroencephalogram signal. The method includes: receiving in real time an electroencephalogram signal data stream collected by an electroencephalographic device; segmenting the electroencephalogram signal data stream in real time via a sliding window approach; obtaining parameter information of the electroencephalographic device, and performing, based on the parameter information, adaptive filtering and artifact removal on a segment of the electroencephalogram signal data stream, the parameter information including a number of channels and a sampling rate; and matching a feature extraction strategy for one or more pre-selected output indicators, and extracting in real time, based on the matched feature extraction strategy, a feature value conforming to the output indicators from the filtered and artifact-removed segment of the electroencephalogram signal data stream from perspectives including time domain analysis, frequency domain analysis, time-frequency domain analysis, and/or nonlinear analysis.

### CROSS-REFERENCE TO RELATED APPLICATIONS

The present disclosure is a continuation of International Application No. PCT/CN2024/140724, filed on Dec. 19, 2024, which claims priority to claims priority to Chinese patent application No. 202311775102.6, titled “METHOD AND SYSTEM FOR REAL-TIME ARTIFACT PROCESSING AND FEATURE EXTRACTION OF ELECTROENCEPHALOGRAM SIGNAL” filed with China National Intellectual Property Administration on Dec. 21, 2023, Chinese patent application No. 202311844300.3, titled “HUMAN-FACTOR INTELLIGENCE-BASED ELECTROENCEPHALOGRAM SIGNAL PROCESSING METHOD AND APPARATUS” filed with China National Intellectual Property Administration on Dec. 28, 2023, and Chinese patent application No. 202311823752.3, titled “HUMAN-FACTOR INTELLIGENCE-BASED ELECTROENCEPHALOGRAM SIGNAL CORRECTING METHOD AND APPARATUS, filed with China National Intellectual Property Administration on Dec. 27, 2023, the entire contents of which are incorporated herein by reference.

### FIELD

The invention relates to the technical field of electroencephalogram signal processing, in particular to a method and a system for real-time artifact processing and feature extraction of an electroencephalogram signal.

### BACKGROUND

An electroencephalogram signal captures electrical activity of the cerebral cortex, featuring a millisecond-level temporal resolution. This allows it to extract a wide variety of feature values and exhibit high responsiveness. In addition, with the development of sensor technology, portable electroencephalographic devices have become gradually diversified and are widely used in fields such as biofeedback, emotion detection, state recognition, and a brain-computer interface. The electroencephalogram signal originates from synchronous discharge of a large number of neuronal postsynaptic membranes in the cerebral cortex. The electroencephalogram signal may be categorized into an evoked electroencephalogram signal and a spontaneous electroencephalogram signal based on whether an external stimulus is applied. The former is induced by the external stimulus and exhibits obvious time-locking and phase-locking characteristics. Through superposition, Event-Related Potentials (ERPs) may be obtained to reflect brain cognitive processes. The spontaneous electroencephalogram signal, characterized by a specific physiological rhythm, comprises five frequency bands: δ, θ, α, β, and γ. Each frequency band has its own scalp distribution and physiological significance, and may be modulated by the stimulus to exhibit an increase or a decrease in energy within a specific frequency band, thereby reflecting a physical and mental state of an individual.

However, electroencephalographic data is significantly affected by noise and an artifact, and has complex and diverse feature values that reflect different aspects of the electroencephalogram signal. Therefore, accurately analyzing the data and extracting valid feature values are crucial for improving application sensitivity. This necessitates that researchers possess extensive experience in electroencephalographic data processing and solid coding skills, which may create a barrier to wide spread cross-disciplinary application of an electroencephalographic device. Also, most existing applications are developed based on a single type of feature value, lacking an ability to integrate multi-dimensional data. In this way, a loss of representation of brain activities information and a reduction in a data volume can occur, lowering accuracy of applications in fields such as emotion detection, state recognition, and the brain-computer interface. The electroencephalogram signal is highly random, with a weak amplitude, and is extremely vulnerable to contamination by irrelevant noise, resulting in various artifacts, such as an electrooculographic artifact, an electromyographic artifact, a sweat artifact, and mains interference. Therefore, an electroencephalogram signal directly recorded from a scalp electrode often cannot accurately represent a brain neural signal. Preprocessing and denoising of the collected raw electroencephalographic data are essential to minimize or eliminate an impact of these artifacts as much as possible. Artifacts in the electroencephalogram signal may be roughly divided into two categories: a physiological artifact and a non-physiological artifact. The physiological artifact is usually caused by activities of body parts near the head, with the most common one being an electrophysiological signal generated by eye blinks, eye movements, tongue movements, heartbeats, respiration, muscle activity, and sweat gland excitation. For example, an electrooculographic signal is generated by potential differences caused by movement of dipoles between the cornea and the retina during eye movements. These potential differences may change an electric field around the eyes, affecting a scalp electric field. An electrocardiographic artifact is usually generated when an ipsilateral ear, which is far from an electrode, is used as a reference. A frontalis muscle artifact is mainly caused by forced eye closure. The non-physiological artifact usually originates from external environmental interference, with the most common being the mains interference. The non-physiological artifact may also be generated when there is poor contact between the electrode site and the scalp or when an electroencephalographic recording system malfunctions. For example, when the electrode moves, an electric double layer is disturbed, generating a direct current. Loose wires or loose circuit boards are also important causes of the non-physiological artifact, which may lead to a loss of some signals and an intermittent malfunction.

In the related art, there is a lack of technical solutions for real-time processing of the electroencephalogram signal. In addition, there is a lack of integrated and automated solutions for processing electroencephalogram signals from different electroencephalographic devices.

### SUMMARY

To solve the aforementioned problems in the related art, the present disclosure provides a method and a system for real-time artifact processing and feature extraction of an electroencephalogram signal, aiming to eliminate or mitigate one or more defects existing in the related art.

In an aspect, the present disclosure provides a method for real-time artifact processing and feature extraction of an electroencephalogram signal. The method includes: receiving in real time an electroencephalogram signal data stream collected by an electroencephalographic device; segmenting the electroencephalogram signal data stream in real time via a sliding window approach; obtaining parameter information of the electroencephalographic device, and performing, based on the parameter information, adaptive filtering and artifact removal on a segment of the electroencephalogram signal data stream, the parameter information comprising a number of channels and a sampling rate; and matching a feature extraction strategy for one or more pre-selected output indicators, extracting in real time, based on the matched feature extraction strategy, a feature value conforming to the output indicators from the filtered and artifact-removed segment of the electroencephalogram signal data stream from perspectives comprising time domain analysis, frequency domain analysis, time-frequency domain analysis, and/or nonlinear analysis.

In another aspect, the present disclosure provides a system for real-time artifact processing and feature extraction of an electroencephalogram signal. The system includes: a user configuration module configured to receive one or more pre-selected output indicators, and obtain parameter information of an electroencephalographic device, the parameter information including a number of channels and a sampling rate; a built-in processing module configured to perform, based on the parameter information, adaptive filtering and artifact removal on a segment of the electroencephalogram signal data stream, match a feature extraction strategy for the one or more pre-selected output indicators, and extract in real time, based on the matched feature extraction strategy, a feature value conforming to the output indicators from the filtered and artifact-removed segment of the electroencephalogram signal data stream, frequency domain analysis, time-frequency domain analysis, and/or nonlinear analysis; and a data transmission module configured to receive in real time the electroencephalogram signal data stream collected by the electroencephalographic device and transmit the feature value that is extracted in real time and conforms to the output indicators.

In another aspect, the present disclosure provides an apparatus for real-time artifact processing and feature extraction of an electroencephalogram signal. The apparatus includes: a memory having a computer instruction stored therein; and a processor configured to execute the computer instruction stored in the memory. The apparatus is configured to, when the computer instruction is executed by the processor, implement the method according to any one of the above embodiments.

In another aspect, the present disclosure provides a computer-readable storage medium, having a computer program stored thereon. The program is configured to, when executed by a processor, implement the method according to any one of the above embodiments.

With the method and the system for real-time artifact processing and feature extraction of the electroencephalogram signal provided in the present disclosure, adaptive filtering and artifact removal can be performed on the electroencephalogram signal data stream based on the parameter information such as the number of channels and the sampling rate included in the electroencephalographic device. In addition, a feature extraction strategy is matched based on pre-selected output indicators to extract in real time a feature value conforming to the output indicators. On the one hand, real-time artifact removal and feature extraction for the electroencephalogram signal can be realized. On the other hand, corresponding feature values for different types of electroencephalographic devices and different output indicators can be integrally and automatically extracted, facilitating subsequent analysis and processing of electroencephalographic data.

### BRIEF DESCRIPTION OF THE DRAWINGS

The accompanying drawings described herein are provided to further understand the present disclosure and constitute a part of the present disclosure. They are not intended to limit the present disclosure.

FIG. 1 is a flowchart of a method for real-time artifact processing and feature extraction according to an embodiment of the present disclosure.

FIG. 2 is a workflow diagram of a system for real-time artifact processing and feature extraction according to an embodiment of the present disclosure.

FIG. 3 is a schematic diagram of a built-in algorithm module of a system for real-time artifact processing and feature extraction according to an embodiment of the present disclosure.

FIG. 4 is a flowchart of an artifact removal method using an FastICA+GFP combined technology according to an embodiment of the present disclosure.

FIG. 5 is a schematic diagram of a structure of a convolutional neural network model according to an embodiment of the present disclosure.

FIG. 6 is a schematic flowchart of a human-factor intelligence-based electroencephalogram signal processing method according to an embodiment of the present disclosure.

FIG. 7 is a schematic diagram of a detailed architecture of a convolutional neural network model according to an embodiment of the present disclosure.

FIG. 8 is a schematic diagram of a structure of an electronic device according to an embodiment of the present disclosure.

### DETAILED DESCRIPTION

To make purposes, technical solutions, and advantages of the present disclosure clearer, the present disclosure will be further described in detail below in conjunction with embodiments and accompanying drawings. Herein, illustrative embodiments of the present disclosure and descriptions thereof are used to explain the present disclosure, but are not intended as limitations on the present disclosure. It should also be noted herein that, to avoid obscuring the present disclosure due to unnecessary details, only structures and/or processing steps closely related to the solutions according to the present disclosure are shown in the accompanying drawings, while other details that are less relevant to the present disclosure are omitted.

It should be emphasized that the term “include/comprise” as used herein refers to the presence of features, elements, steps, or components, but does not exclude the presence or addition of one or more other features, elements, steps, or components.

It should also be noted herein that, unless otherwise specified, the term “connected” as used herein may refer not only to direct connection, but also to indirect connection with an intermediate substance present.

The embodiments of the present disclosure will be described below with reference to the accompanying drawings. In the accompanying drawings, the same reference numerals represent the same or similar components, or the same or similar steps.

### First Embodiment

To address the problems in an existing method for real-time artifact processing and feature extraction of an electroencephalogram signal, the present disclosure provides a method for real-time artifact processing and feature extraction of an electroencephalogram signal.

The problems in the existing method include: (1) inability to perform real-time data analysis: existing electroencephalogram data analysis software such as EEGLAB and MNE cannot receive and process data in real time, and can only perform post-hoc analysis on the electroencephalogram data, typically in a segment of several minutes, and cannot output data in a segment of several seconds, making it difficult to meet application requirements in fields such as real-time state recognition and emotion detection; (2) difficulties in data analysis and export: the electroencephalogram data has a complex multi-dimensional structure. Feature extraction for each feature type relies on a substantial coding foundation, and it is difficult to export a feature value for each subject and each channel individually, resulting in extremely inconvenient data application; (3) low degree of automation: an existing artifact removal method mostly adopts Independent Component Analysis (ICA). On one hand, this method has high requirements for researchers' experience, requiring researchers to able to identify and remove an artifact such as an electrooculographic artifact by themselves. This process cannot be automated and is time-consuming and labor-intensive; (4) inability to meet processing needs of diverse devices: ICA processing requires a sufficient number of electroencephalogram channels and a sufficiently long duration of the electroencephalogram data. Therefore, an artifact cannot be removed from single-channel or fewer-channel electroencephalogram data, nor can the processing be performed on data segments of several milliseconds. Existing research on automatic artifact detection and removal is limited to either multi-channel data or single-channel data, lacking an integrated method; (5) high application development requirements: applying an analyzed feature value in various fields requires independent development of a real-time API or a data transmission function such as TCP/UDP, complicating the application process.

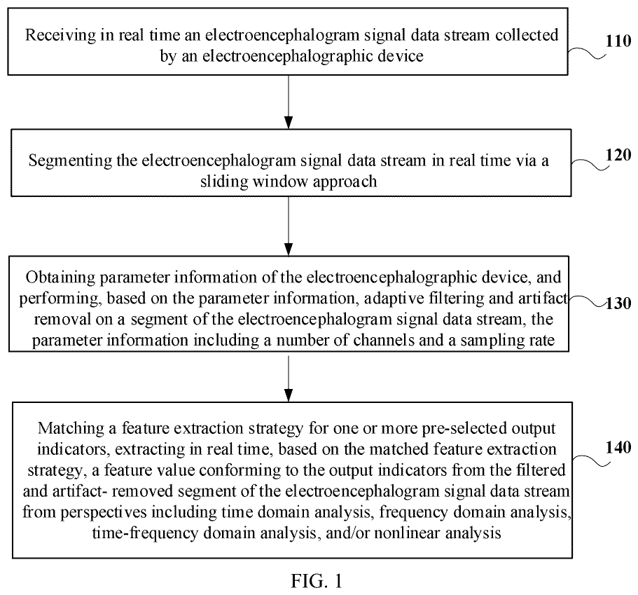

FIG. 1 is a flowchart of a method for real-time artifact processing and feature extraction according to an embodiment of the present disclosure. The method includes operations at blocks:

At S110, an electroencephalogram signal data stream collected by an electroencephalographic device is received in real time.

In a specific implementation process, a system for real-time artifact processing and feature extraction of the electroencephalogram signal supports multiple protocol transmission methods such as Bluetooth and Lab Streaming Layer (LSL). The system can receive a data stream from the electroencephalographic device in real time, read and cache the real-time collected data according to a user configuration, and employ an adaptive sliding window paradigm to perform real-time noise reduction, artifact removal, and feature extraction.

In the specific implementation process, the electroencephalographic device is a device configured to record brain activity. The device includes electrodes placed on the scalp, an amplifier for amplifying weak signals captured by the electrodes to a level processable by a computer, and a computer for converting the signal output by the amplifier into a signal in a graphic format or a digital format. Moreover, the collected electroencephalogram signal data stream may include a predetermined number of channels, and electroencephalogram signal data streams from different channels may be distinguished by using electrode placement regions as classification labels.

At S120, the electroencephalogram signal data stream is segmented in real time via a sliding window approach.

In the specific implementation process, segmenting the electroencephalogram signal data stream in real time using the sliding window approach refers to dividing the electroencephalogram signal data stream into multiple consecutive segments of a fixed length, with each segment being called a window. First, the electroencephalogram signal data stream is arranged in a chronological order. Then, a window length set. Starting from the beginning, the consecutive segments of the electroencephalogram signal data stream, each having the specified window length, are sequentially extracted as windows. This step is repeated until the entire electroencephalogram signal data stream is divided into multiple windows.

At S130, parameter information of the electroencephalographic device is obtained, and adaptive filtering and artifact removal are performed on the segments of the electroencephalogram signal data stream based on the parameter information. The parameter information includes a number of channels and a sampling rate.

In the specific implementation process, the system conducts research on types of electroencephalographs used in different scenarios such as a standard laboratory, biofeedback, an emotional brain-computer interface, and a medical-type brain-computer interface. Personalized filtering solutions are provided for high-precision, high-resolution, and high-density gel electrode electroencephalograph devices, portable wearable high-density saline (semi-dry) electrode electroencephalograph devices, and portable wearable dry electrode electroencephalograph devices of different forms (with different numbers of channels). By integrating conventional frequency-domain filtering, wavelet denoising, and an innovative hybrid method such as deep learning, blind source separation, and machine learning, fully automated signal denoising and artifact removal are realized. A user only needs to perform simple configuration, and an entire artifact removal process is completed automatically, without writing a large amount of code.

The filtering includes one or more of low-pass filtering, high-pass filtering, band-pass filtering, and notch filtering. The artifact removal includes one or more of Independent Component Analysis (FastICA), Global Field Power (GFP) analysis, blind source signal separation, and SVM-based artifact removal.

At S140, a feature extraction strategy is matched for one or more pre-selected output indicators, and based on the matched feature extraction strategy, a feature value conforming to the output indicators is extracted in real time from the filtered and artifact-removed segments of the electroencephalogram signal data stream from perspectives including time domain analysis, frequency domain analysis, time-frequency domain analysis, and/or nonlinear analysis.

In the specific implementation process, feature extraction algorithms for electroencephalogram signals from multiple sources may be integrated, including an algorithm for calculating feature values corresponding to time domain, frequency domain, time-frequency domain, nonlinearity dynamics, and brain functional connectivity, thereby providing diverse and highly sensitive feature values. Through the receipt of user configuration to select an output indicator, a feature extraction strategy, filtering processing, etc., the system adapts to different scenarios.

A protocol supporting transmission of each of the electroencephalogram signal data stream collected by the electroencephalographic device and the extracted feature value, includes one or more of TCP protocol, UDP protocol, wireless Bluetooth protocol, MQTT protocol, RS-232/RS-485 protocol, and LSL protocol.

With the method and the system for real-time artifact processing and feature extraction of the electroencephalogram signal provided in the present disclosure, the adaptive filtering processing and the artifact removal processing can be performed on the electroencephalogram signal data stream based on the parameter information such as the number of channels and the sampling rate of the electroencephalographic device. In addition, a feature extraction strategy is matched based on the pre-selected output indicators to extract in real time the feature value conforming to the output indicators. On the one hand, real-time artifact removal and feature extraction for the electroencephalogram signal can be realized. On the other hand, corresponding feature values for different types of electroencephalographic devices and different output indicators can be extracted in an integrated and automated manner, facilitating subsequent analysis and processing of the electroencephalographic data.

In some embodiments of the present disclosure, prior to receiving the electroencephalogram signal data stream and performing artifact processing and feature extraction, the method further includes: obtaining device information of the electroencephalographic device from which the electroencephalogram signal data stream originates. The device information includes the number of channels and the sampling rate.

Further, in a specific embodiment of the present disclosure, the method further includes obtaining user configuration for real-time artifact processing and feature extraction. The user configuration includes selection of a time window size of a sliding window, a filtering strategy, an artifact removal strategy, a feature value output indicator, and a protocol supporting feature value transmission.

In some embodiments of the present disclosure, in a scenario of identifying an anxiety state based on the electroencephalogram signal, an energy value of a frequency band, an energy value of θ frequency band, and an energy value of γ frequency band are used as the output indicators. The artifact removal process employed includes independent component analysis processing and global field power analysis processing. In a scenario of prosthesis control based on the electroencephalogram signal, a feature value corresponding to output indicators of event-related synchronization and desynchronization of μ rhythm and β rhythm is extracted in real time from the filtered and artifact-removed segment of the electroencephalogram signal data stream from the perspective of time-frequency domain analysis based on the matched feature extraction strategy.

In some embodiments of the present disclosure, the extracting in real time, based on the matched feature extraction strategy, the feature value conforming to the output indicators from the filtered and artifact-removed segment of the electroencephalogram signal data stream from the perspectives comprising time domain analysis, frequency domain analysis, time-frequency domain analysis, and/or nonlinear analysis includes: (1) from the perspective of time domain analysis, extracting in real time, based on a statistical algorithm or an Hjorth algorithm, one or more of a mean value, a variance, a standard deviation, a kurtosis, a skewness, and an autocorrelation coefficient from the filtered and artifact removed segment of electroencephalogram signal data stream; (2) from the perspective of frequency domain analysis, extracting in real time an energy value and/or a power value from the segment of the filtered and artifact-removed electroencephalogram signal data stream based on any one of fast Fourier transform algorithm, a periodogram method, a Welch method, a multi-window method, and an autoregressive model; (3) from the perspective of time-frequency domain analysis, extracting in real time, based on short-time Fourier transform or continuous wavelet transform, the feature value from the filtered and artifact-removed segment of the electroencephalogram signal data stream; and (4) from the perspective of nonlinear analysis, extracting in real time, based on recursive variable analysis and complexity, one or more of Shannon entropy, approximate entropy, sample entropy, and permutation entropy from the filtered and artifact-removed segment of the electroencephalogram signal data stream.

By adopting the embodiments of the present disclosure, multiple feature extraction methods can be integrated to meet the electroencephalographic feature extraction requirements in different scenarios and extract corresponding feature values.

In some embodiments of the present disclosure, the performing, based on the parameter information, adaptive filtering and artifact removal on the segment of the electroencephalogram signal data stream includes: when a number of channels of the electroencephalogram signal collected by the electroencephalographic device is smaller than a predetermined threshold, using a pre-trained neural network model to perform adaptive filtering and artifact removal on the segment of the electroencephalogram signal data stream. The neural network model is obtained through supervised learning and training using large-scale dataset of electroencephalogram signal containing different types of artifacts and/or having different signal-to-noise ratios. For example, the channel number threshold may be any one of 3, 5, or 10. The pre-trained model is used to obtain a target result based on the extracted feature value.

By adopting the embodiments of the present disclosure, training can be performed based on historical data for different electroencephalogram signals, which makes the artifact removal and feature extraction of the electroencephalogram signal more representative and obtaining purer electroencephalographic data.

In another aspect, the present disclosure provides a system for real-time artifact processing and feature extraction of an electroencephalogram signal. The system includes: (1) a user configuration module configured to receive one or more pre-selected output indicator, and to obtain parameter information of an electroencephalographic device, the parameter information including a number of channels and a sampling rate; (2) a built-in processing module configured to: perform, based on the parameter information, adaptive filtering and artifact removal on segments of the electroencephalogram signal data stream; match a feature extraction strategy for the one or more pre-selected output indicators, and extract in real time, based on the matched feature extraction strategy, a feature value conforming to the output indicators from the processed segments of the electroencephalogram signal data stream from perspectives including time domain analysis, frequency domain analysis, time-frequency domain analysis, and/or nonlinear analysis; and (3) a data transmission module configured to receive in real time the electroencephalogram signal data stream collected by the electroencephalographic device and transmit the feature value that is extracted in real time and conforms to the output indicators.

The system for real-time artifact processing and feature extraction of the electroencephalogram signal provided in the present disclosure is based on an integrated electroencephalographic data analysis platform ErgoLAB. The platform aims to address numerous difficulties and shortcomings in existing electroencephalographic data analysis, making real-time processing and feature extraction of electroencephalographic data more efficient, automated, and convenient. ErgoLAB refers to an ErgoLAB human-machine-environment synchronization cloud platform. Based on a cloud architecture and synchronization technology, this cloud platform is professionally used for “human-centered” synchronous collection and quantitative analysis of multi-modal data, emphasizing intelligence and wearability. Combining VR (Virtual Reality) and simulation technology, light environment simulation technology, brain cognitive neuroscience and electrophysiology technology, eye tracking technology, movement capture technology, behavior analysis technology, facial expression and state recognition technology, etc., an interactive influence relationship among human, a machine, and an environment is objectively and quantitatively analyzed, enhancing depth of longitudinal research and extensiveness of horizontal research. The platform is compatible with many scientific research devices in the fields of cognitive neuroscience, human factors engineering, and artificial intelligence, providing a complete solution for fields such as human factors engineering and ergonomics, architecture and environmental behavior, human-computer interaction and artificial intelligence, psychology and cognitive science, and traffic driving behavior research.

The system mainly comprises three parts: the user configuration module, the built-in algorithm module, and the data transmission module. (1) the user configuration module may allow the user to select a denoising method, an artifact removal algorithm, a feature value type, and a data transmission method as desired, and may also allow input of information such as the sampling rate and number of channels of the used device. The system may automatically recommend an algorithm (information such as the number of channels and the sampling rate influences an applicable artifact removal algorithm and a type of an extractable feature value), to realize personalized data processing settings. (2) The built-in algorithm module is a core part of this toolbox. Firstly, the module integrates a variety of classic and advanced noise and artifact removal methods, such as filtering, wavelet denoising, FastICA+GFP, SOBI+SVM, and complex neural networks and convolutional neural networks, aiming to automatically remove an artifact such as an electrooculographic artifact. Secondly, the built-in algorithm module also includes various feature value extraction algorithms, covering multi-dimensional feature value calculation in the time domain, the frequency domain, the time-frequency domain, and the nonlinear analysis, making feature extraction of electroencephalogram data simple and efficient. Also, the built-in algorithm module also designs different feature fusion solutions for a combination of different feature values, for example, using CNN to fuse a spatial-domain electroencephalographic feature and a frequency-domain electroencephalographic feature. (3) The data transmission module may provide a variety of transmission methods, including TCP transmission, UDP transmission, and API transmission, offering convenient output and application interfaces for the analyzed feature value. Through appropriate filtering, noise in the electroencephalographic data can be effectively reduced. A common filtering method includes the low-pass filtering, the high-pass filtering, the band-pass filtering, and the notch filtering. A low-pass (high-pass) filter may allow a signal below (above) than a predetermined cut-off frequency to pass through while attenuating or eliminating a signal above (below) than this frequency. A band-pass filter may allow only a specific frequency to pass through, while effectively suppressing signals at other frequencies. A notch filter may attenuate or eliminate a signal component at a specific frequency. A filter is intuitive and easy to understand, capable fast real-time processing. However, it cannot fully adapt to complex signal structures and spectral characteristics. For example, the filter cannot remove an artifact such as the electrooculographic artifact that is confounded with the genuine signal.

The wavelet denoising is a commonly used signal denoising method based on a principle of wavelet transform. By decomposing the signal into frequency bands at different scales, and performing noise estimation and elimination according to a statistical characteristic of the signal, thereby reducing a noise component in the signal. The wavelet denoising may capture both a local feature and a global feature of the signal, adaptively select a threshold for noise estimation and elimination, and effectively retain a detailed feature of the signal. However, its computational complexity is relatively high, and the selection of wavelet basis function and the threshold significantly impacts the noise removal effect. Empirical Mode Decomposition (EMD) is an adaptive signal decomposition method that decomposes a nonlinear and non-stationary signal into a set of Intrinsic Mode Functions (IMF). This method constructs local extreme points of the signal, obtains an upper envelope and a lower envelope through interpolation, calculates a local mean line, and subtracts the local mean from the signal to obtain a detail curve. This process is repeated until the detail curve meets a condition of IMF, i.e., a number of maximum points and a number of minimum points on the curve is equal to each other or differs by at most one. After an iterative process, the finally obtained IMFs are added together to approximately reconstruct an original signal. The Empirical Mode Decomposition has advantages of adaptability, multi-scale decomposition, and no need for a predetermined parameter. However, the Empirical Mode Decomposition is highly dependent on the data, may suffer from a mode mixing problem, and may be sensitive to selection of an initial point and the interpolation method. Independent Component Analysis (ICA) is used to decompose a mixed signal into independent components. It assumes that the mixed signal is a linear combination of source signals, and utilizes a statistical characteristic of independence, to separate the source signals.

By adopting the system for real-time artifact processing and feature extraction of the electroencephalogram signal, researchers and application developers may no longer be limited by a conventional electroencephalographic data analysis software. They can process real-time data quickly and accurately to extract an effective feature value, providing a novel solution for widespread applications of the electroencephalographic device in the fields such as biofeedback, emotion detection, state recognition, and the brain-computer interface. Development of this system may greatly improve an efficiency and an automation of electroencephalographic data analysis, and thus bring broader development prospects to electroencephalographic research and applications.

In yet another embodiment of the present disclosure, the system for real-time artifact processing and feature extraction of the electroencephalogram signal includes a real-time electroencephalographic data collection and analysis module, an intelligent noise and artifact removal module, an automated multi-dimensional feature extraction module, and a data transmission module. In an exemplary embodiment of the present disclosure, (1) the real-time electroencephalographic data collection and analysis module of the system can receive a data stream from the electroencephalographic device in real time, and support multiple protocol transmission methods such as wireless Bluetooth and Lab Streaming Layer (LSL). According to user configuration, it reads and caches the real-time collected data, and adopts the adaptive sliding window paradigm to perform real-time denoising, artifact removal, and feature extraction. (2) the intelligent noise and artifact removal module of the system investigates the type of the electroencephalograph devices used in different scenarios such as the standard laboratory, the biofeedback, the emotional brain-computer interface, and the medical-type brain-computer interface. It designs personalized filtering solution for high-precision, high-resolution, and high-density gel electrode electroencephalograph devices, portable wearable high-density saline (semi-dry) electrode electroencephalograph devices, and portable wearable dry electrode electroencephalograph devices of different forms (with different numbers of channels). By integrating conventional frequency-domain filtering, wavelet denoising, and innovative hybrid method such as deep learning, blind source separation, and machine learning, fully automated signal denoising and artifact removal are realized. A user only needs to perform simple configuration, and an entire artifact removal process can be automatically completed without writing a large amount of code. (3) Based on extensive research of literature and algorithms in relevant fields, the system integrates feature value calculation algorithms corresponding to the time domain, the frequency domain, the time-frequency domain, the nonlinear analysis, and brain functional connectivity. The feature values are diverse and highly sensitive. (4) the data transmission module of the system is compatible with multiple data transmission protocols such as TCP, UDP, the wireless Bluetooth, MQTT, and RS-232/RS-485, realizing convenient data transmission between different systems, different devices, and different applications. Researchers only need to select as desired without writing code.

Further, a combination of FastICA and GFP may be used to remove an electrooculographic artifact from an electroencephalogram signal with 8 or more channels. The general procedure is as follows: for example, the FastICA algorithm is used to calculate an independent component of each lead, respectively, while a GFP value of the selected N leads is calculated. Then, a correlation coefficient between each independent component and the GFP is calculated, and an independent component corresponding to a maximum correlation coefficient is output. This maximum component is excluded, and other components are reconstructed to obtain a clean signal. This method is not applicable to an electroencephalographic device with fewer channels in a prefrontal lobe. In this case, the system may automatically recommend a pre-trained neural network model to achieve end-to-end electroencephalogram signal denoising. When training such model, a large number of electroencephalogram signals containing different artifacts (such as the electrooculographic artifact and the electromyographic artifact) and having different signal-to-noise ratios are first input. Supervised learning is performed using these electroencephalogram signals to obtain optimal parameters, which are then deployed in the system for real-time or offline noise identification and removal.

In some embodiments of the present disclosure, the automated multi-dimensional feature extraction module includes feature extraction from multiple perspectives. Specific examples are listed as follows.
(1) From the perspective of time-domain analysis, time-domain analysis reveals variation of amplitude over time, typically calculated through statistical method for various feature values.

TABLE 1
Examples of time-domain feature indicators for an electroencephalogram signal

| Indicator | | |
|---|---|---|
| Name | Description | Meaning |
| Kurtosis | Kurtosis is a statistic describing a | High kurtosis indicates presence of a |
| | shape of signal data distribution, | sharp peak or a very flat region in |
| | usually calculated using a | the signal, while low kurtosis |
| | fourth-order central moment | indicates a smoother signal |
| Skewness | Skewness is a statistic used to | Reflects non-uniformity of brain |
| | describe a degree to which the | activity |
| | signal data distribution is skewed | |
| | to the left or the right | |
| Mean | An arithmetic mean of the data | Represents an overall voltage level |
| | | of the electroencephalogram signal |
| Standard | A degree of dispersion of a data | Indicates overall stability of the |
| Deviation | point from the mean | electroencephalogram signal |
| Event-related | Refers to a time delay of an | Reflects a speed of brain processing |
| potential | electroencephalogram response | |
| latency | triggered by a certain event or | |
| | stimulus | |
| Event-related | Amplitude refers to a peak of the | Indicates the intensity of the |
| potential | signal or a peak-to- trough height | cognitive processing induced by the |
| amplitude | of the signal | stimulus |

(2) From the perspective of frequency-domain analysis, frequency-domain analysis describes distribution of information such as energy and a phase of the electroencephalogram signal across frequency points, which can reflect a cognitive state of an individual. A common method for frequency-domain analysis includes Fourier transform, periodogram method, Welch method, multi-window method, autoregressive model, etc.
The Fourier transform is a basic frequency-domain analysis method, which is used to convert a time-domain signal into a frequency-domain signal. This method may decompose the signal into amplitude and phase information of different frequency components. The Fourier transform is usually suitable for a stationary signal. The periodogram method is a method used to study a periodic component in the signal. The periodogram method includes a tool such as an autocorrelation function, a cross-correlation function, and power spectral density, which can be used to identify periodicity in the signal. The Welch method is a signal processing method that divides a signal into multiple overlapping windows and calculates a power spectral density for each window. Then, these power spectral densities estimates are averaged to obtain a final estimate, to reduce a variance of the estimate. The multi-window method involves using different types of window functions, such as rectangular window, Hamming window, Hanning window, to analyze a spectral characteristic of the signal. Different window functions are suitable for different applications and signal types. The autoregressive model is a method used to model time-series data, and usually used to estimate a frequency component of the signal. The autoregressive model includes an autoregressive (AR) model and an autoregressive moving average (ARMA) model.
(3) From the perspective of spectral analysis, EEG spectral analysis includes a classic direct method (the periodogram method) and the most commonly used improved direct method (the Welch method). An idea of the Welch method is as follows: a signal of length N is divided into L segments, a length of each segment of data is M, and thus N=LM; then, a window function w is applied to each segment of data, and a power spectrum of each segment of data is calculated; finally, power spectra of all segments of data are averaged to obtain a power spectrum of an entire signal. The segments of data may overlap, and the window function w may be any type of window such as the Hanning window or the Hamming window.
(3)

TABLE 2
Examples of frequency-domain feature indicators
for an electroencephalogram signal

| Indicator | | |
|---|---|---|
| Name | Description | Meaning |
| Delta | Typically, in a range of | A higher Δ may indicate a deep |
| | 0.5 Hz to 4 Hz. | sleep state or a pathological |
| | | condition. |
| Theta | Typically, between 4 Hz | Related to a relaxed state, creative |
| | and 8 Hz. | thinking, and shift in attention. An |
| | | increased θ wave may indicate an |
| | | inattention or a relaxed state. |
| Alpha | Typically, in a range of 8 | Occurs during rest or when sitting |
| | Hz to 13 Hz. | quietly with eyes closed, and |
| | | related to relaxation and attentional |
| | | rest. A decreased α wave may |
| | | indicate an increase in alertness. |
| Beta | Typically, between 13 | Related to alertness, recognition, |
| | Hz and 30 Hz. | and attention. An increased β may |
| | | indicate a state of excitement or |
| | | cognitive activity. |
| Gamma | Typically, above 30 Hz. | A γ wave is related to high-level |
| | | cognition, learning, and information |
| | | processing. An increased γ may |
| | | indicate high-level cognitive |
| | | activity. |
| Power Peak | Refers to a maximum | The power peak may provide |
| | power value in the | information about a main frequency |
| | frequency spectrum, | characteristic of |
| | usually corresponding to | electroencephalographic activity. |
| | a specific frequency | |
| | band. | |
| α/β | A ratio between the α | A higher α/β ratio may indicate a |
| | wave and the β wave, | relaxed state. |
| | usually used to explore a | |
| | balance between | |
| | alertness and a relaxed | |
| | state. | |
| θ/β | A ratio between the θ | A higher θ/β ratio may indicate |
| | wave and the β wave, | inattention. |
| | used to evaluate | |
| | attention and alertness. | |
| (α + θ)/β | A ratio between a sum | Provides comprehensive |
| | of the α wave and the θ | information about a relaxed state |
| | wave to the β wave. | and attention |
| (α + θ)/(α + β) | A ratio between a sum | Reflects an overall balance of |
| | of the α wave and the θ | electroencephalographic brave |
| | wave to a sum of the α | |
| | wave and the β wave. | |
| θ/(α + β) | A ratio between the θ | Evaluates a balance between |
| | wave to the sum of the α | attention and a resting state |
| | wave and the β wave. | |
| SMR (db) | Represents a | SMR is related to sensorimotor |
| | sensorimotor rhythm, | integration and control. |
| | typically in a range of | |
| | 12 Hz to 15 Hz. | |

(4) From the perspective of time-frequency analysis, a power value of the signal at each specific time and frequency point in the time-frequency domain is estimated. When performing spectral analysis, time information is retained. The spectral analysis is typically performed using methods such as short-time Fourier transform or continuous wavelet transform. A result of time-frequency analysis is used to indicate an increase or decrease in signal power within a specific frequency band in a corresponding ROI region after a certain stimulus appears.
(5) From the perspective of nonlinear dynamics analysis, a result of nonlinear dynamics analysis is used to reflect changes in a dynamic characteristic of the brain, including indicators such as complexity, entropy, and Lyapunov exponent. Complexity measures an information capacity of an electroencephalogram signal segment, reflecting a potential activity characteristic of a neuron and representing a speed at which a new pattern appears in a time series as a length of the series increases. The complexity indicator includes Lempel-Ziv complexity, Lempel-Ziv permutation complexity, etc. An entropy value indicates a degree of disorder in the electroencephalogram signal. Different entropy algorithms describe the information capacity from different perspectives. A decrease in entropy value means a reduction in an information interaction ability within the brain. The entropy indicator includes time-domain-based Shannon entropy, approximate entropy, sample entropy, and permutation entropy. Time-frequency-based entropy includes wavelet entropy and Hilbert-Huang spectrum entropy. A maximum Lyapunov exponent may quantitatively describe an average divergence rate of adjacent orbits in a phase space and how small initial state perturbation in the system gradually amplify over time.
(6) The brain functional connectivity indicator is used to evaluate information connectivity between brain regions. Each part of the brain has a unique function in human behavior. Even the simplest task requires collaboration among multiple brain regions. A common indicator includes a coherence-based indicator, a phase synchronization-based indicator, a generalized synchronization-based indicator, and a Granger causality-based indicator.

Based on this, the system for real-time artifact processing and feature extraction of the electroencephalogram signal provided by the present disclosure may restrict selectable feature types based on information such as channels and sampling rate of the device set by the user. Also, the system may also receive a further instruction from the researcher to screen feature values based on the researcher's experimental needs. In addition, the extracted feature value supports one-click export.

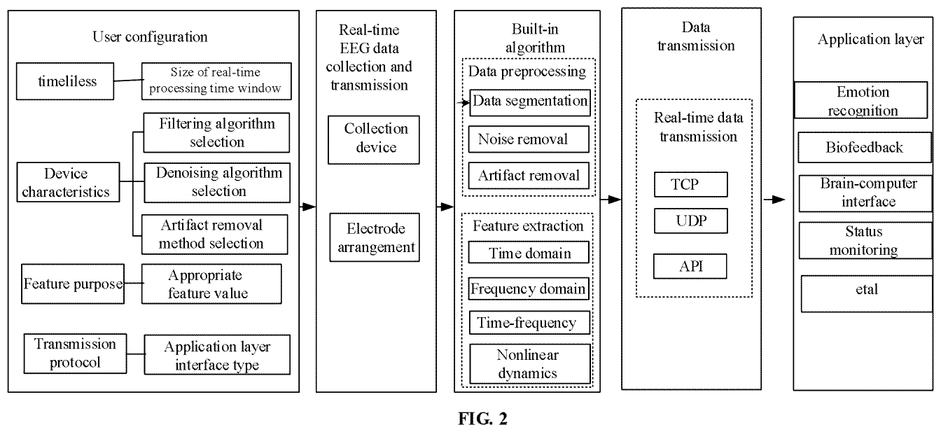

FIG. 2 is a workflow diagram of a system for real-time artifact processing and feature extraction according to an embodiment of the present disclosure. First, the system receives user configuration. In terms of timeliness, the user configuration includes a size of a real-time processing time window. In terms of a device characteristic, the user configuration involves selection of a filtering algorithm, a denoising algorithm, and an artifact removal method. In terms of feature usage, the user configuration includes selection of an appropriate feature value that needs to be extracted. In terms of transmission protocols, the user configuration involves selection of an application layer interface type. After the user configuration is determined, real-time electroencephalogram signal data transmission is performed. Electrodes are arranged at different positions on a head of a subject. The collection device transmits the collected electroencephalogram signal to the system provided by the present disclosure. Through a built-in algorithm, data preprocessing is performed on a collected original electroencephalogram signal, including data segmentation, noise removal, and artifact removal. Then, the feature value is extracted from one or more of perspectives including the time domain, frequency domain, time-frequency domain, and nonlinear dynamics. Extraction is performed in accordance with a pre-selected appropriate feature value. Subsequently, the extracted feature value is transmitted in real time via TCP, UDP, or API. Finally, at an application layer, operations such as emotion recognition, biofeedback, brain-computer interface-related operations, and state monitoring are performed based on different application scenarios of the electroencephalogram signal.

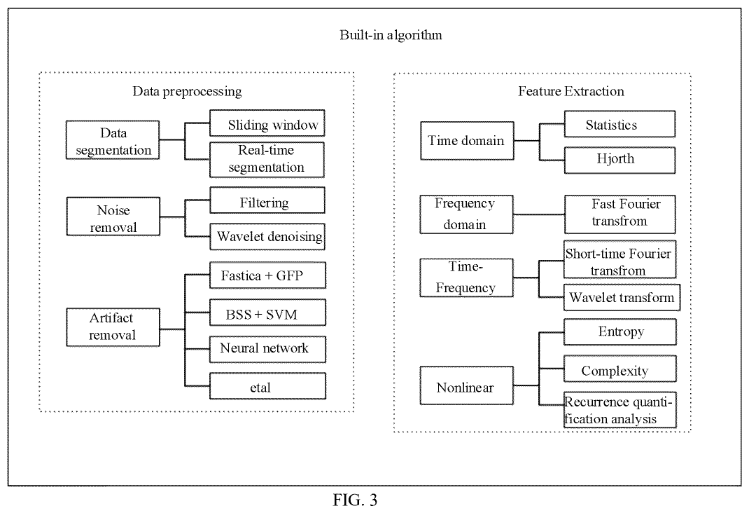

FIG. 3 is a schematic diagram of a built-in algorithm module for a system for real-time artifact processing and feature extraction according to an embodiment of the present disclosure. On the basis of FIG. 2, FIG. 3 lists different built-in algorithms that may be used for data preprocessing and feature extraction. For example: (1) for data segmentation, sliding window and real-time segmentation are adopted; (2) for noise removal, filtering and/or a wavelet denoising algorithm are used; (3) for artifact removal, algorithms such as FastICA+GFP, BSS+SVM, and neural networks are applied; (4) for time-domain feature extraction, a statistical method or Hjorth algorithm are adopted; (5) for frequency-domain feature extraction, fast Fourier transform is used; (6) for time-frequency feature extraction, short-time Fourier transform or wavelet transform algorithms are adopted; (7) for feature extraction from a nonlinear perspective, algorithms such as entropy, complexity, or recurrence quantification analysis algorithm are used. After removing a physiological artifact and a non-physiological artifact such as electrooculogram, electromyogram, power line interference, noise, and drift by methods like noise reduction, a clean electroencephalogram signal is obtained. Then, features of the electroencephalogram signal in multiple dimensions such as time domain, frequency domain, time-frequency domain, and nonlinear domain can be extracted by methods such as fast Fourier transform, short-time Fourier transform, and wavelet transform.

A specific embodiment of the present disclosure is used in the field of emotion recognition using an electroencephalogram signal. By collecting an electroencephalogram signal to identify an anxious state, meditation relaxation is guided. Anxiety may affect life and work status of an individual. In the case of persistent anxiety, a person's immune system may also be affected, leading to pain and diseases. In a state of anxiety, energy values in α frequency band and θ frequency band of the brain may decrease. After mindfulness training such as meditation relaxation, γ frequency band tends to strengthen. Real-time visualization of the energy values in α frequency band, θ frequency band, and γ frequency band guides anxious individuals in meditation relaxation.

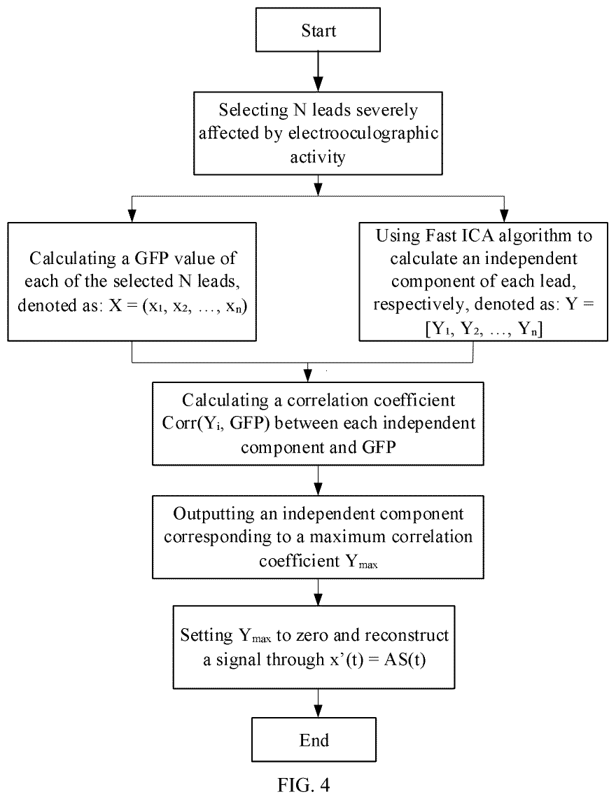

In an exemplary embodiment of the present disclosure, a detailed process in this specific embodiment includes the following steps.
Step 1: user configuration. On a configuration interface, a real-time data transmission method is selected as wireless network transmission (which may be wireless Bluetooth transmission, the selection with priority given to transmission efficiency, and the present disclosure is not limited in this regard). The parameter information of the device is inputted with a number of channels of 12 leads (a channel in the electroencephalogram signal is called “lead”, so it may be expressed as “12 leads”) and a sampling rate of 250 Hz. The ErgoLAB real-time artifact removal and feature extraction system recommends high-pass and low-pass filtering of 0.1-70 Hz and notch filtering of 50 Hz based on the device information. During artifact removal, the system recommends a multi-channel artifact detection algorithm such as FastICA+GFP (as illustrated in the figure below). All types of extractable feature values are displayed, from which the user selects the energy value of the α frequency band, the energy value of the θ frequency band, and the energy value of the γ frequency band under frequency-domain features as output indicators. The time window is set to 2 seconds, and a feature value transmission method adopts the TCP protocol. The time window refers to a dataset within a predetermined time range. In a stream processing scenario, data exists in the form of a continuous stream. Data is continuously generated without a clear beginning or end. Use of the time window can split the data stream into a plurality of data blocks. Subsequent processing of the data stream is performed based on the time window. This is a conventional setting for frequency-domain feature analysis of the electroencephalogram signal. FIG. 4 is a flowchart of an artifact removal method using a FastICA+GFP combined technology according to an embodiment of the present disclosure.
Step 2: feature value extraction. Real-time transmitted electroencephalogram data is temporarily stored. using sliding window approach, a time window of several second preceding the current time point is taken for noise and artifact removal (specific method vary depending on different artifact removal algorithms). After obtaining a clean signal, the period of 2 seconds preceding the current time point are taken for frequency-domain feature extraction, with a step length size of 1 second.
Step 3: a biofeedback system design. The system has a built-in breathing meditation training course, through which the individual may learn a breathing relaxation method. 5 minutes of data is collected from the individual in a quiet and stress-free state as a baseline. A visualization design is made based on energy values of the three frequency bands: the α frequency band, the θ frequency band, and the γ frequency band. A visualized graphic is a flower bud, where the flower bud opens under low anxiety and closes in high anxiety.

TABLE 3
Anxiety level classification table based on different
frequency bands of an electroencephalogram signal

| Frequency | | | Flower |
|---|---|---|---|
| band | Judgment | Rule | Bud |
| α frequency band | Below baseline as T | One T represents | An opening degree of |
| θ frequency band | Below baseline as T | anxiety | the flower bud |
| γ frequency band | Above baseline as T | Two Ts represent | gradually decreases |
| | | relatively anxious | from one T to three Ts |
| | | Three Ts represent | |
| | | severe anxiety | |

Step 4: real-time feedback adjustment. The ErgoLAB real-time artifact processing and feature extraction system transmits the extracted α, θ and γ frequency band energy values every 2 seconds to the biofeedback system in real time via the TCP protocol. The flower bud on the biofeedback system interface begins to change dynamically. The individual needs to use the learned breathing relaxation method to make the flower bud bloom as much as possible.

A specific embodiment of the present disclosure is used in the scenario of collecting an electroencephalogram signal for controlling a prosthetic limb. By collecting an electroencephalogram signal in real time from a right-limb disabled individual during motor imagery of forward, backward, leftward, and rightward, a time-frequency analysis method is used to extract the corresponding event-related synchronization and event-related desynchronization (ERS and ERD) of μ rhythm and β rhythm. Then, a machine learning method is adopted for classification. The prosthetic limb is controlled based on a classification result.

In an exemplary embodiment of the present disclosure, a detailed process of technical implementation in this embodiment includes the following steps.

### Step 1: User Configuration

The user selects the real-time data transmission method as wireless Bluetooth transmission on the configuration interface and inputs the parameter information of the device. The number of channels is 32 leads. The sampling rate is 512 Hz. According to the device information, the ErgoLAB real-time artifact removal and feature extraction system recommends high-pass and low-pass filtering of 0.1-70 Hz and notch filtering of 50 Hz. For the artifact removal, an SOBI+SVM algorithm is recommended. Time-frequency analysis is selected, with the focus on the μ rhythm spanning 8 Hz to 13 Hz, and the β rhythm spanning 13 Hz to 30 Hz.

### Step 2: Feature Value Extraction

The real-time transmitted electroencephalogram data is temporarily stored. By using the sliding window approach, the time window of several seconds preceding the current time point is taken for noise and artifact removal (specific method vary depending on different artifact removal algorithms), to obtain a clean signal. Then, according to an event code indicating the beginning of motor imagery, time period of 1000 ms after the beginning of motor imagery and time period of 200 ms before the beginning of motor imagery is taken as the event-related time window and the baseline, respectively, for short-time Fourier transform, to extract ERS and ERD of μ rhythm and β rhythm.

### Step 3: Classification

Label setting: according to the requirements during imagery, the data is marked. The marked data is divided into a training set and a test set. A 4-classification model is created. The model is created on the training set. Parameters of the model is adjusted on a validation set. A training result is evaluated using the test set. A model with the best accuracy is selected for real-time prosthetic arm control.

### Step 4: Real-Time Control

The ERS and ERD extracted from the μ rhythm and the β rhythm in real time are sent to the model by means of the TCP protocol. A classification result of the model is outputted for controlling the prosthetic limb.

A specific embodiment of the present disclosure is applied in a scenario of collecting the electroencephalogram signal to evaluate a comprehensive efficiency of an operator. In this scenario, the electroencephalogram signals are collected from the operator when performing tasks of different difficulties. Diverse feature values are extracted from dimensions such as time domain, frequency domain, time-frequency, and nonlinear analysis using feature extraction algorithms. The feature values are marked in combination with questionnaire scale information. Machine learning methods are used to classify electroencephalogram data under tasks of different difficulties, classify mental fatigue, a workload, and a stress state of the operator, and the classification results for these three dimensions are weighted to calculate the comprehensive efficiency of the operator, to reduce occurrence of accidents.

In an exemplary embodiment of the present disclosure, a detailed process of technical implementation in this embodiment includes the following steps.

### Step 1: User Configuration

The user selects the real-time data transmission method as wireless Bluetooth transmission on the configuration interface and inputs the parameter information of the device. The number of channels is 64 leads. The sampling rate is 1024 Hz. According to the device information, the ErgoLAB real-time artifact removal and feature extraction system recommends high-pass and low-pass filtering of 0.1-70 Hz and filtering of 50 Hz notch. For artifact removal, a wavelet denoising algorithm is recommended. Diverse feature value types are selected, including mean, variance, kurtosis, skewness, etc. in time domain; power spectral density, frequency band energy, etc. in frequency domain; short-time Fourier transform, etc. in time-frequency domain; sample entropy, Lyapunov exponent, etc. in nonlinear analysis. The time window is set to 2 S. The feature value transmission method is set to TCP protocol. The Lyapunov exponent represents a numerical feature of an average exponential divergence rate of adjacent orbits in a phase space. The Lyapunov exponent is also known as an Lyapunov characteristic exponent, which is one of several numerical features used to identify a chaotic motion.

The time-frequency analysis is joint analysis of the electroencephalogram signal in the time domain and the frequency domain. The time-frequency analysis aims to study dynamic changes of the signal across time and frequency. For example, the event-related synchronization (ERS) of μ (sensorimotor) rhythm occurs at an electrode in a prefrontal lobe and a parietal lobe (a sensorimotor region) within a period of time after the motor imagery task.

The nonlinear analysis is analysis of a nonlinear dynamic characteristic in the electroencephalogram signal. The electroencephalogram signal has complex nonlinear dynamic characteristics. The nonlinear analysis aims to reveal nonlinear interactions, nonlinear dynamic behaviors, and chaotic characteristics in the signal. Commonly used nonlinear analysis methods include phase space reconstruction, Lyapunov exponent, nonlinear prediction, etc.

### Step 2: Feature Value Extraction

The real-time transmitted electroencephalogram data is temporarily stored. By using a sliding window approach, the time window of several seconds preceding the current time point is taken for noise and artifact removal ((specific method vary depending on different artifact removal algorithms), to obtain a clean signal. Then, a period of 2S immediately preceding the current time point is taken for diverse feature extraction.

### Step 3: Classification and Evaluation

The electroencephalogram data is marked based on questionnaire scale data. The data is divided into a training set and a test set. Separate binary classification models are created for load, fatigue, and stress, respectively. These models are trained using the training set, optimized and fine-tuned via methods such as cross-validation, and evaluated on the test set. This process is repeated multiple times. Models with superior evaluation indicators such as accuracy and recall rate are selected.

An expert evaluation method is used to evaluate a weight of each model, which are denoted as W1, W2, and W3, respectively. A total score of comprehensive efficiency F is calculated as F=(W1×load (0, 100)+W2×fatigue (0, 100)+W3×stress (0, 100))/3.

### Step 4: Real-Time Feature Transmission and Status Warning

The diverse feature values extracted in real time are sent to the models via TCP protocol, and the classification results of the models are outputted respectively. The score of comprehensive efficiency is calculated based on the results and the weights of the models. A higher score of comprehensive efficiency indicates a poorer working state of the operator. When the score of comprehensive efficiency is higher than 80 points, a warning is triggered.

With the method and the system for real-time artifact processing and feature extraction of the electroencephalogram signal provided in the present disclosure, based on the parameter information such as the number of channels and the sampling rate included in the electroencephalographic device, adaptive filtering and artifact removal can be performed on the electroencephalogram signal data stream. A feature extraction strategy is matched based on the pre-selected output indicators to extract in real time a feature value conforming to the output indicators. On one hand, real-time artifact removal and feature extraction for the electroencephalogram signal can be realized. On the other hand, corresponding feature values for different types of electroencephalographic devices and different output indicators can be extracted in an integrated and automated manner, facilitating subsequent analysis and processing of electroencephalographic data. The method provided in the present disclosure can support the export data channel by channel and feature by feature, making data application more convenient. Through a variety of artifact removal methods, a high degree of automated processing can be achieved, eliminating the need for researchers to spend time and effort on identifying a crisis component, and placing no demands on the researchers' experience. This method integrates different artifact removal methods, enabling artifact removal for not only electroencephalogram data with a single channel or a small number of channels, but also for electroencephalogram data of multiple-channels.

Corresponding to the above method, the present disclosure also provides an apparatus for real-time artifact processing and feature extraction of an electroencephalogram signal. The apparatus includes a computer device. The computer device includes a processor and a memory. The memory has a computer instruction stored therein. The processor is configured to execute the computer instruction stored in the memory. When the computer instruction is executed by the processor, the apparatus implements the steps of the above-mentioned method.

### Second Embodiment

The technical solution according to embodiments of the present disclosure mainly addresses a deficiency of existing technologies in temporal feature information extraction capability when using a neural network model to extract information from an electroencephalogram (EEG) signal. In an exemplary embodiment of the present disclosure, for the electroencephalogram signal applied in a technical field such as the brain-computer interface, classification and other operations may be mainly performed based on temporal feature information and spatial feature information in the electroencephalogram signal. The temporal feature information mainly refers to information obtained by analyzing a waveform morphology of the electroencephalogram signal, including an amplitude, a slope, a peak, a trough, etc. The spatial feature information mainly refers to spatial distribution information of a multi-channel electroencephalogram signal, which can be obtained by studying a correlation between channels to describe brain activities. Fully extracting the above-mentioned temporal feature information and spatial feature information in the neural network model can help have a more comprehensive and accurate understanding of brain activities, which is conducive to subsequent operations such as classification.

In the related art, there is provided a convolutional neural network model capable of processing an electroencephalogram signal, known as an EEGNet model. Although the model may also extract temporal feature information and spatial feature information, for one thing, convolutional layers in a second feature extraction module (block 2) and a third feature extraction module (block 3) of the model extract redundant temporal feature information, leading to feature redundancy between the convolutional layers, and limiting a final classification effect of the model. This results in a weak capability in extracting temporal feature information.

To address the above technical problem, the embodiments of the present disclosure provide a new convolutional neural network model architecture, and use this model architecture to provide a human-factor intelligence-based electroencephalogram signal processing method. The new convolutional neural network model architecture includes a first feature extraction module, a second feature extraction module, a third feature extraction module, and a classification module that are connected in sequence. Each of the first feature extraction module, the second feature extraction module, and the third feature extraction module includes a temporal convolution kernel for extracting temporal feature information. The second feature extraction module further includes a spatial convolution kernel for extracting spatial feature information. By setting the temporal convolution kernels responsible for temporal feature information extraction in all three feature extraction modules, more comprehensive extraction of the temporal feature information can be achieved, enhancing the capability of temporal feature information extraction. Further, different types of convolutional layers may be set in different feature extraction modules such that characteristics of different types of convolutional layers can be utilized. In this way, not only the extracted temporal feature information can be continuously optimized but also the feature redundancy problem existing in a current EEGNet model can be solved. A specific architecture of the convolutional neural network model and the electroencephalogram signal processing method will be described in detail in subsequent embodiments of the present disclosure.

The technical solution of the embodiment of the present disclosure will be described in detail below with reference to the accompanying drawings. FIG. 5 is a schematic diagram of a structure of a convolutional neural network model according to an embodiment of the present disclosure. As illustrated in FIG. 5, the convolutional neural network model includes a first feature extraction module 11, a second feature extraction module 12, a third feature extraction module 13, and a classification module 14 that are connected in sequence. An electroencephalogram signal collected by the electroencephalogram collection device may be directly inputted to the first feature extraction module 11, or inputted to the first feature extraction module 11 after preprocessing such as denoising, and then sequentially processed by the subsequent modules. Each of the first feature extraction module 11, the second feature extraction module 12, and the third feature extraction module 13 includes a temporal convolution kernel for extracting temporal feature information. The second feature extraction module 12 further includes a spatial convolution kernel for extracting spatial feature information.

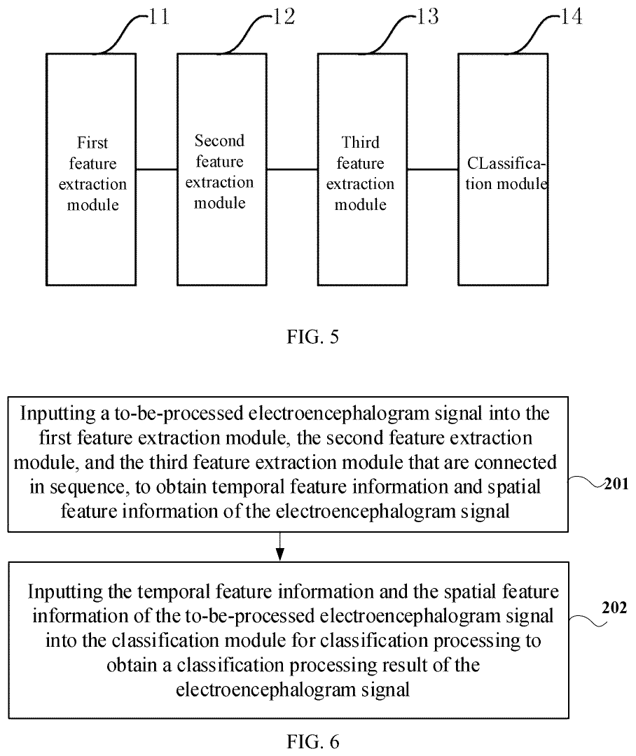

FIG. 6 is a schematic flowchart of a human-factor intelligence-based electroencephalogram signal processing method according to an embodiment of the present disclosure. This method may be performed on an electronic device. The electronic device includes, but is not limited to, a server, a local computer. As illustrated in FIG. 6, the method includes operations at blocks.

At 101, a to-be-processed electroencephalogram signal is inputted into the first feature extraction module, the second feature extraction module, and the third feature extraction module that are connected in sequence, to obtain temporal feature information and spatial feature information of the electroencephalogram signal.

The to-be-processed electroencephalogram signal in this step may be collected by an electroencephalogram signal collection device. Based on different application scenarios of the embodiments of the present disclosure, the scenario may be an online processing scenario. In this case, an electroencephalogram signal collected by the electroencephalogram signal collection device can be directly inputted into the convolutional neural network model according to the embodiments of the present disclosure for processing. Alternatively, the scenario may be an offline processing scenario. In this case, the collected electroencephalogram signal can be stored in a storage module in advance, and then inputted into the convolutional neural network model for processing only when the electroencephalogram signal is processed according to the technical solution of the present disclosure. The above to-be-processed electroencephalogram signal may be directly collected by the electroencephalogram signal collection device, or may be an electroencephalogram signal obtained after preprocessing a directly collected signal. The above preprocessing includes, but is not limited to, denoising. In addition, the above electroencephalogram signal collection device may be composed of a plurality of signal detection probes to collect a multi-channel electroencephalogram signal.

Each of the first feature extraction module, the second feature extraction module, and the third feature extraction module in the embodiments of the present disclosure is provided with a convolution kernel for extracting temporal feature information or spatial feature information. Therefore, when the to-be-processed electroencephalogram signal is inputted into the first feature extraction module, the temporal convolution kernel therein may extract the temporal feature information from the electroencephalogram signal to obtain a first feature map, which contains the temporal feature information. This first feature map is further inputted into the second feature extraction module. The spatial convolution kernel in the second feature extraction module may extract the spatial feature information from the first feature map. The temporal convolution kernel in the second feature extraction module may further extract the temporal feature information. This temporal feature information is a preliminary optimization of the temporal feature information in the first feature map. The second feature extraction module may output a second feature map that includes the spatial feature information and the preliminarily optimized temporal feature information. After the second feature map is further inputted into the third feature extraction module, the temporal convolution kernel in the third feature extraction module may further perform more refined optimization on the above preliminarily optimized temporal feature information to obtain finally optimized temporal feature information. Finally, the third feature module may output the third feature map. The third feature map may include the above spatial feature information and the finally optimized temporal feature information.

It should be understood that the to-be-processed electroencephalogram signal in the second embodiment of the present disclosure may correspond to the filtered and artifact-removed electroencephalogram signal data stream in the above first embodiment. By using the second embodiment, spatial feature information and temporal feature information of the filtered and artifact-removed electroencephalogram signal data stream may be extracted.

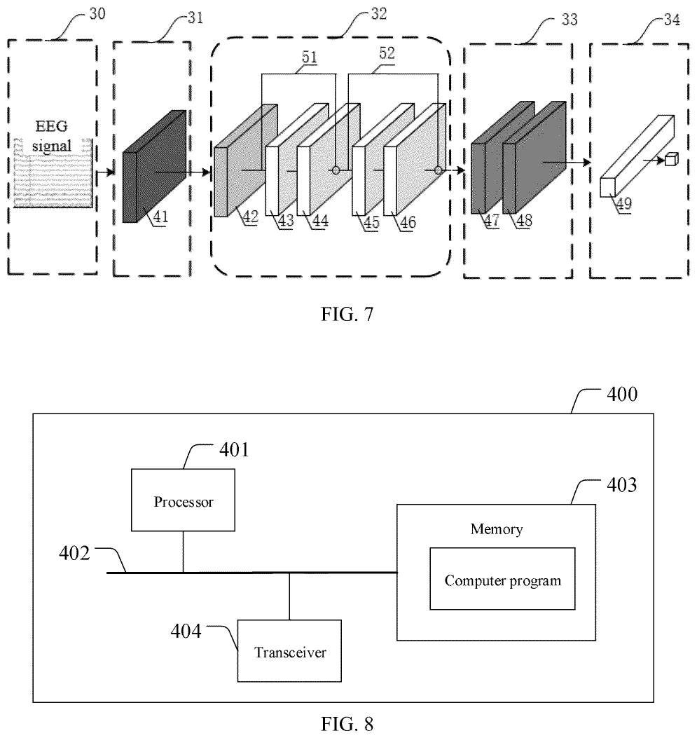

A specific structure and function of the first feature module, a specific structure and function of the second feature module, and a specific structure and function of the third feature module that are involved in the embodiments of the present disclosure will be described in detail in the subsequent embodiment illustrated in FIG. 7.

At 102, the temporal feature information and the spatial feature information of the to-be-processed electroencephalogram signal are inputted into the classification module for classification processing to obtain a classification processing result of the electroencephalogram signal.

In this step, after obtaining the temporal feature information and the spatial feature information of the electroencephalogram signal, the temporal feature information and the spatial feature information may be inputted into the classification module for classification processing. A specific classification result included in the classification processing result may be set as desired. For example, to understand an electroencephalogram signal controlling different parts of the body, the classification result may include the control of corresponding different body parts. In some embodiments, the classification module in this step may include a fully connected layer and a normalization function. The fully connected layer may map feature information to respective classification results. The normalization function may be a softmax function, which may obtain probabilities corresponding to different categories in different classification results. A sum of the probabilities for all classification results is 1.

In the above embodiments of the present disclosure, each of the first feature extraction module, the second feature extraction module, and the third feature extraction module in the convolutional neural network model includes a temporal convolution kernel for extracting the temporal feature information, which can enhance extraction of the temporal feature information from the electroencephalogram signal. Also, the spatial convolution kernel in the second feature extraction module realizes extraction of the spatial feature information from the electroencephalogram signal. Therefore, more comprehensive and accurate extraction of feature information from the electroencephalogram signal is realized, which is beneficial to improving accuracy of the classification result obtained by the classification module based on the temporal feature information and the spatial feature information.

FIG. 7 is a schematic diagram of a specific architecture of a convolutional neural network model according to an embodiment of the present disclosure. As illustrated in FIG. 7, the convolutional neural network model includes an input module 30, a first feature extraction module 31, a second feature extraction module 32, a third feature extraction module 33, and a classification module 34. The embodiments of the present disclosure will provide a detailed description and explanation of each of the above modules.

The input module 30 is mainly configured to receive a to-be-processed electroencephalogram signal. In an exemplary embodiment of the present disclosure, the to-be-processed electroencephalogram signal may include a multi-channel electroencephalogram signal collected by an electroencephalogram collection device. In addition, the electroencephalogram signal may be preprocessed such as denoised when necessary. After receiving an input electroencephalogram signal, the electroencephalogram signal may be transmitted to the first feature extraction module 31 for processing. In the embodiments of the present disclosure, the electroencephalogram signal may be treated as an image signal for processing.

In some embodiments, the first feature extraction module 31 includes at least one depthwise separable convolutional layer 41. Each of the at least one depthwise separable convolutional layer includes a first temporal convolution kernel for extracting temporal feature information. In this case, in the first feature extraction module, the step of processing may include: inputting the to-be-processed the electroencephalogram signal electroencephalogram signal into the first feature extraction module to obtain a first feature map output from the first feature extraction module. The first feature map includes the temporal feature information extracted by the first temporal convolution kernel.

In the embodiments of the present disclosure, one depthwise separable convolutional layer 41 may be provided. A depth of this depthwise separable convolutional layer 41 may be set as desired. For example, the depth may be set to values such as 4 or 8. In some embodiments, a size of the first temporal convolutional kernel may be set to (1, a), where a is a positive integer greater than 1. That is, a height and a width of the first temporal convolutional kernel are set to 1 and a, respectively. By the first temporal convolutional kernel, feature extraction may be performed in a width direction of an electroencephalogram signal image to obtain the temporal feature information in the electroencephalogram signal.

In some embodiments, a Batch Normalization (BN) layer and an activation function may be further provided in the first feature extraction module 31. The activation function may be an RELU activation function. The BN layer and RELU activation function may be sequentially disposed at an output end of the depthwise separable convolutional layer.

In the embodiments of the present disclosure, the depthwise separable convolutional layer 41 used in the first feature extraction module 31 inherently has advantages of using fewer parameters and enabling separation of channels and regions. Experimental verification has shown that using the depthwise separable convolutional layer 41 alone to extract the temporal feature information of the electroencephalogram signal yields better results than using various other types of convolutional layers.

In the embodiments of the present disclosure, the second feature extraction module 32 may include at least one standard convolutional layer 42 and at least two residual blocks that are connected in sequence. The at least one standard convolutional layer 42 includes a spatial convolution kernel for extracting spatial feature information. Each of the at least two residual blocks includes a second temporal convolution kernel for extracting temporal feature information. In the embodiments of the present disclosure, the first feature extraction module 31 performs feature extraction and obtains the first feature map. Therefore, a specific processing for the second feature extraction module 32 is as following: inputting the first feature map into the second feature extraction module 32 to obtain a second feature map output from the second feature extraction module 32. The second feature map includes the spatial feature information extracted by the spatial convolution kernel and a first optimized temporal feature information extracted by the second temporal convolution kernel.

In some embodiments, one standard convolutional layer 42 may be included. A depth of the standard convolutional layer 42 may be set as desired. For example, the depth may be set to values such as 8 or 16. A size of the spatial convolution kernel in the standard convolutional layer 42 is (channel, 1), where “channel” refers to the number of channels of the electroencephalogram signal. A height and a width of the spatial convolution kernel are set to “channel” and 1, respectively. By the spatial convolution kernel, feature extraction can be performed in a height direction of the electroencephalogram signal image to obtain the spatial feature information in the electroencephalogram signal.

In the embodiments of the present disclosure, as illustrated in FIG. 7, at least one residual block of the second feature extraction module 30 may include a first residual block and a second residual block that are connected in sequence. The first residual block includes a first convolutional layer 43, a second convolutional layer 44, and a first skip connection 51 that are connected in sequence. The first skip connection 51 is configured to add input information of the first convolutional layer 43 to output information of the second convolutional layer 44 as output information of the first residual block. The second residual block includes a third convolutional layer 45, a fourth convolutional layer 46, and a second skip connection 52 that are connected in sequence. The second skip connection 52 is configured to add input information of the third convolutional layer 45 to output information of the fourth convolutional layer 46 as output information of the second residual block. The first convolutional layer 43, the second convolutional layer 44, the third convolutional layer 45, and the fourth convolutional layer 46 each includes the second temporal convolution kernel for extracting temporal feature information.

In some embodiments, the first convolutional layer 43, the second convolutional layer 44, the third convolutional layer 45, and the fourth convolutional layer 46 may be standard convolutional layers. Their depths can be set as desired, for example, to be 4, 8, 16, or 32, etc. The first convolutional layer 43 and the second convolutional layer 44 may be set to have the same depth, and the third convolutional layer 45 and the fourth convolutional layer 46 may be set to have the same depth. Alternatively, all four convolutional layers can have the same depth. In some embodiments, a size of the second temporal convolution kernel in each of the first convolutional layer and the second convolutional layer is (1, a), where a is the positive integer greater than 1. A size of the second temporal convolution kernel in each of the third convolutional layer and the fourth convolutional layer is (1, b), where b is the positive integer greater than 1. The four convolution kernels are set as a and b in a width direction, respectively, enabling them to obtain the temporal feature information in the electroencephalogram signal.

In some embodiments, the Batch Normalization (BN) layer and the activation function may be further provided in the second feature extraction module 32. The activation function may be the RELU activation function. The BN layer and RELU activation function may be sequentially disposed at an output end of each of the above convolutional layers.

Further, a first average pooling layer may be provided at an output end of the second feature extraction module 32. A pooling kernel size adopted by the first average pooling layer may be (1, 4), to perform average pooling on the second feature map obtained by processing each of the above convolutional layers, and obtain a pooled second feature map.

The second feature extraction module provided in the embodiments of the present disclosure has a strong ability to learn temporal feature information and expands a parameter space of the network model. Compared with the first feature extraction module, the two residual blocks in the second feature extraction module may extract more refined temporal feature information.

In the embodiment of the present disclosure, the third feature extraction module 33 may include at least one mixed dilated convolutional layer. Each of the at least one mixed dilated convolutional layer includes a third temporal convolution kernel for extracting temporal feature information. The third feature extraction module 33 in this embodiment may input the second feature map into the third feature extraction module 33 to obtain a third feature map output from the third feature extraction module 33. The third feature map includes the spatial feature information extracted by the spatial convolution kernel and a second optimized temporal feature information extracted by the third temporal convolution kernel.

In some embodiments, the third feature extraction module 33 may include a first mixed dilated convolutional layer and a second mixed dilated convolutional layer that are connected in sequence. That is, the third feature extraction module 33 may include two mixed dilated convolutional layers. The mixed dilated convolutional layer may combine dilated convolution and standard convolution, and is a type of Hybrid Dilated Convolution (HDC). The mixed dilated convolutional layer can process a multi-scale feature, ensure spatial resolution of features, and improve a generalization ability of the model. A depth of the mixed dilated convolutional layer may be set as desired. As the depth increases, a size of a receptive field of the third temporal convolution kernel in the mixed dilated convolution may gradually increase. Use of the mixed dilated convolutional layer in this embodiment can enhance a representational ability of the convolutional neural network model and effectiveness of the receptive field.

Specifically, in an exemplary embodiment of the present disclosure, a size of the third temporal convolution kernel may be set to (1, c), where c is the positive integer greater than 1. The convolution kernel is set to c in the width direction, enabling it to obtain temporal feature information in the electroencephalogram signal. Moreover, since mixed dilated convolution is used in this embodiment, multiple ablation experiments conducted in the embodiments of the present have verified that the technical solution of the present disclosure can extract more comprehensive temporal feature information, improving accuracy of the final model classification result.

In some embodiments, a Batch Normalization layer may be further disposed at an output end of the first mixed dilated convolutional layer 47 and an output end of the above second mixed dilated convolutional layer 48.

In the embodiments of the present disclosure, the depthwise separable convolutional layer 41, the first convolutional layer 43, the second convolutional layer 44, the third convolutional layer 45, the fourth convolutional layer 46, the first mixed dilated convolutional layer 47, and the second mixed dilated convolutional layer 48 are all provided with temporal convolution kernels capable of extracting temporal feature information. However, the implementation forms of these convolution kernels differ, allowing them to extract temporal feature information from different dimensions. In addition, since the above convolutional layers are arranged in sequence, the temporal feature information can be continuously refined and optimized, ultimately yielding more comprehensive and accurate temporal feature information and enhancing the extraction of temporal feature information from the input electroencephalogram signal.

In some embodiments, for the embodiment shown in FIG. 7, the depths of the depthwise separable convolutional layer 41, the standard convolutional layer 42, the first convolutional layer 43, the second convolutional layer 44, the third convolutional layer 45, the fourth convolutional layer 46, the first mixed dilated convolutional layer 47, and the second mixed dilated convolutional layer 48 are configured in an inverted bottleneck structure. That is, the depths of the depthwise separable convolutional layer 41 and the second hybrid dilated convolutional layer 48 are set as a minimum value, for example, 4, while the depths of intermediate convolutional layers are set as other values greater than 4, for example, 8 or 16. Alternatively, in some embodiments, for the intermediate convolutional layers, the depths of the standard convolutional layer 42 and the first mixed dilated convolutional layer 47 that are located at two ends are set to a smaller value, for example, 16, and the depths of the first convolutional layer 43, the second convolutional layer 44, the third convolutional layer 45, the fourth convolutional layer 46 in the middle are set to 32 to form an inverted bottleneck structure. This setting of depths of the convolutional layers helps reduce a number of parameters used.

In the embodiments of the present disclosure, an ELU activation function and a second average pooling layer that are connected in sequence may be further disposed at an output end of the third feature extraction module 33. The second average pooling layer may use pooling kernel size of (1, 8). Experimental verification has shown that using the ELU activation function in the embodiments of the present disclosure achieves better accuracy than using the ReLU activation function.

In addition, in the three feature extraction modules according to the embodiments of the present disclosure, the use of activation layers between multiple convolutional layers is reduced. For example, a single ELU activation function is set in the third feature extraction module 33, which is conducive to obtaining more accurate feature information and improving the accuracy of the final classification result.

In the embodiments of the present disclosure, temporal feature information is first extracted in the first feature extraction module, followed by spatial feature information extraction in the second feature extraction module. Therefore, the technical solution according to the embodiments of the present disclosure essentially involves extracts temporal feature information first and then spatial feature information. Experimental verification shows that this solution yields better results than the technical solution in the EEGNet model, which extracts spatial feature information first and then temporal feature information.

In the embodiments of the present disclosure, the classification module 34 may further include a fully connected layer 49 and a loss function. The fully connected layer 49 may map the temporal feature information and the spatial feature information extracted by the above feature extraction modules to a respective classification result, to achieve a goal of obtaining the classification result based on the temporal feature information and the spatial feature information. A normalization function may adopt the Softmax function, which may be used to limit a sum of probabilities of all classification results to be 1.

The convolutional neural network model according to the above embodiments of the present disclosure is a model specially designed for decoding and classifying an electroencephalogram signal. This model may be pre-trained, and then the pre-trained convolutional neural network model may be used for classification and prediction of the electroencephalogram signal.

The convolutional neural network model according to the embodiments of the present disclosure may be applied to classification and prediction of left-hand and right-hand motor imagery. A test example for classification and prediction using an existing collected dataset of left-hand and right-hand motor imagery is provided below. When comparing with the classification result of the EEGNet model in the related art, it was found that the proposed model has better advantages in terms of the accuracy of classification result and other aspects, and the differences observed across different subjects are statistically significant (p<0.05). In the above motor imagery dataset, an input image may be in a 4-dimensional format. For example, the input image may be represented as (288, 1, 22, 1000). After processing by each feature extraction module of the convolutional neural network model according to the above embodiments, the finally output feature map may be represented as (288, A, 1, 30). In addition, a padding mode of each convolutional layer in this embodiment may be “SAME.” Therefore, a number of a data point in a fourth dimension remains unchanged after processing by each convolutional layer. Execution steps of a specific embodiment of the present disclosure are as follows.
Step 1: a 4-dimensional electroencephalogram signal is inputted into the first feature extraction module. A size of convolution kernel of the depthwise separable convolutional layer in the first feature extraction module is (1, a). A depth of a convolution filter formed by the convolution kernel may be set to be A, with the padding mode as “SAME.” In this case, after processing by the first feature extraction module, the output first feature map may be represented as (288, A, 22, 1000).
Step 2: the first feature map is inputted into the second feature extraction module. A size of a spatial convolution kernel of a standard convolutional layer in the second feature extraction module is (channel, 1), where a value of “channel” is 22. A depth of a convolution filter formed by the convolution kernel may be B. To form the inverted bottleneck structure, a value of B is greater than a value of A, with the padding mode as “SAME.” After processing by this standard convolutional layer, the obtained feature map may be represented as (288, B, 1, 1000), where the channel is equal to 22, spatial feature information of 22 channels is extracted, and then the 22 channels are compressed into 1. For the two residual blocks, a size of a convolution kernel of a first convolutional layer and a size of a convolution kernel of a second convolutional layer in the first residual block is (1, a). A depth of the formed convolution filter may be B with the padding mode as “SAME.” A size of a convolution kernel of a third convolutional layer and a size of a convolution kernel of a fourth convolutional layer in the second residual block is (1, b). The depth of the formed convolution filter may be B, with the padding mode as “SAME.” After processing by the two residual blocks, a representation form of the feature map remains unchanged and may still be represented as (288, B, 1, 1000). The pooling kernel size of the first average pooling layer is (1, 4). Therefore, a feature map after processing by the first average pooling layer may be represented as (288, B, 1, 250), which may serve as the second feature map.
Step 3: the second feature map is inputted into the third feature extraction module. The third feature extraction module includes two mixed dilated convolutional layers, with the size of the convolution kernel thereof being (1, c). A depth of the convolution filter formed by the first mixed dilated convolutional layer is B, and the depth of the convolution filter formed by the second mixed dilated convolutional layer is A, with the padding mode as same for both. After processing by the first mixed dilated convolutional layer, a representation form of the second feature map remains unchanged. After processing by the second mixed dilated convolutional layer, the feature map may be represented as (288, A, 1, 250). Then, the feature map is processed by the second average pooling layer having a pooling kernel size of (1, 8). The obtained third feature map may be represented as (288, A, 1, 30).
Step 4: 30 data points in the fourth dimension obtained after the above processing are flattened by a fully connected layer. Classification can be performed based on these 30 data points in each batch to obtain a classification result of the dataset, e.g., left hand, right hand, foot, or tongue. This achieves the prediction and classification of the electroencephalogram signal using the convolutional neural network model according to the embodiments of the present disclosure. For the classification result, a predictive ability of the model may be statistically evaluated using a loss value, an accuracy rate, an F1-score, or a confusion matrix.

Corresponding to the above method embodiments, the embodiments of the present disclosure also provide a human-factor intelligence-based electroencephalogram signal processing apparatus, which is executed based on a new convolutional neural network model. The convolutional neural network model includes a first feature extraction module, a second feature extraction module, a third feature extraction module, and a classification module that are connected in sequence. Each of the first feature extraction module, the second feature extraction module, and the third feature extraction module includes a temporal convolution kernel for extracting temporal feature information. The second feature extraction module further includes a spatial convolution kernel for extracting spatial feature information. The apparatus may include an input module and a result acquisition module. The input module is configured to input a to-be-processed electroencephalogram signal into the first feature extraction module, the second feature extraction module, and the third feature extraction module that are connected in sequence, to obtain the temporal feature information and the spatial feature information of the electroencephalogram signal. The result acquisition module is configured to input the temporal feature information and the spatial feature information of the to-be-processed electroencephalogram signal into the classification module for classification processing to obtain a classification processing result of the electroencephalogram signal.

In some embodiments, the first feature extraction module includes at least one depthwise separable convolutional layer. Each of the at least one depthwise separable convolutional layer includes a first temporal convolution kernel for extracting the temporal feature information. The process of inputting the to-be-processed electroencephalogram signal into the first feature extraction module, the second feature extraction module, and the third feature extraction module that are connected in sequence includes: inputting the to-be-processed electroencephalogram signal into the first feature extraction module to obtain a first feature map output from the first feature extraction module. The first feature map includes temporal feature information extracted by the first temporal convolution kernel.

In some embodiments, the first feature extraction module includes one depthwise separable convolutional layer. The first temporal convolution kernel has a size of (1, a), where a is the positive integer greater than 1.

In some embodiments, the second feature extraction module includes at least one standard convolutional layer and at least one residual block that are connected in sequence. In addition, the at least one standard convolutional layer includes the spatial convolution kernel for extracting the spatial feature information. The at least one residual block each includes a second temporal convolution kernel for extracting temporal feature information. The processing of inputting the to-be-processed electroencephalogram signal into the first feature extraction module, the second feature extraction module, and the third feature extraction module that are connected in sequence includes: inputting the first feature map into the second feature extraction module to obtain a second feature map output from the second feature extraction module. The second feature map includes spatial feature information extracted by the spatial convolution kernel and first optimized temporal feature information extracted by the second temporal convolution kernel.

In some embodiments, the second feature extraction module includes a first residual block and a second residual block that are connected in sequence. The first residual block includes a first convolutional layer, a second convolutional layer, and a first skip connection that are connected in sequence. The first skip connection is configured to add input information of the first convolutional layer to output information of the second convolutional layer as output information of the first residual block. The second residual block includes a third convolutional layer, a fourth convolutional layer, and a second skip connection that are connected in sequence. The second skip connection is configured to add input information of the third convolutional layer to output information of the fourth convolutional layer as output information of the second residual block. The first convolutional layer, the second convolutional layer, the third convolutional layer, and the fourth convolutional layer each includes the second temporal convolution kernel.

In some embodiments, a size of the spatial convolution kernel is (channel, 1), where “channel” refers to a number of an output channel of the electroencephalogram signal. A size of the second temporal convolution kernel in the first convolutional layer and the second convolutional layer is (1, a), where a is the positive integer greater than 1. A size of the second temporal convolution kernel in the third convolutional layer and the fourth convolutional layer is (1, b), where b is the positive integer greater than 1.

In some embodiments, the third feature extraction module includes at least one mixed dilated convolutional layer. Each of the at least one mixed dilated convolutional layer includes a third temporal convolution kernel for extracting temporal feature information. The processing of inputting the to-be-processed electroencephalogram signal into the first feature extraction module, the second feature extraction module, and the third feature extraction module that are connected in sequence includes: inputting the second feature map into the third feature extraction module to obtain a third feature map output from the third feature extraction module. The third feature map includes the spatial feature information extracted by the spatial convolution kernel and second optimized temporal feature information extracted by the third temporal convolution kernel.

In some embodiments, the third feature extraction module includes a first mixed dilated convolutional layer and a second mixed dilated convolutional layer that are connected in sequence. The third temporal convolution kernel has a size of (1, c), where c is a positive integer greater than 1.

In some examples, the depth of the depthwise separable convolutional layer, the depth of the standard convolutional layer, the depth of the first convolutional layer, the depth of the second convolutional layer, the depth of the third convolutional layer, the depth of the fourth convolutional layer, the depth of the first mixed dilated convolutional layer, and the depth of the second mixed dilated convolutional layer form an inverted bottleneck structure.

In some embodiments, a first average pooling layer is disposed at an output end of the second feature extraction module. The ELU activation function and the second average pooling layer that are connected in sequence are disposed at an output end of the third feature extraction module.

The human factor intelligence-based electroencephalogram signal processing apparatus according to the embodiments of the present disclosure corresponds to any of the method embodiments shown in FIG. 5 to FIG. 7. It can perform the above method and achieve corresponding technical effects. Detailed descriptions are omitted in the embodiments of the present disclosure. Reference may be made to the above embodiments for specific methods and achieved technical effects.

### Third Embodiment

Brain waves are bioelectrical signals generated when a large number of neurons transmit information during brain activity. By collecting and analyzing an electroencephalogram signal of a target object, a health status of the target object may be identified. The health status is either healthy or unhealthy. The electroencephalogram signal includes: an electroencephalogram signal portion of the target object before being subjected to a predetermined stimulus, and an electroencephalogram signal portion after being subjected to the predetermined stimulus. The electroencephalogram signal portion before being subjected to the predetermined stimulus usually serves as a baseline of the electroencephalogram signal. Generally, the health status of the target object is analyzed and obtained by comparing the electroencephalogram signal portion after being subjected to the predetermined stimulus with this baseline.

In the process of collecting the baseline of the electroencephalogram signal, it is usually necessary for the target object to remain calm, and avoid unexpected stimulus. However, during actual collection, the target object may be subjected to the unexpected stimulus, which may cause the collected electroencephalogram signal portion to be superimposed with an interference signal, causing fluctuations in the baseline of the electroencephalogram signal. If the health status of the target object is determined directly based on the electroencephalogram signal with baseline fluctuations, the accuracy of the determined the health status will be low.

In the related art, to ensure the accuracy of determining the health status of the target object, the electroencephalogram signal of the target object is generally re-collected until a desired electroencephalogram signal that is not subjected to the unexpected stimulus is collected. It can be seen that an efficiency of obtaining the expected electroencephalogram signal in the related art is low. The expected electroencephalogram signal may be used to accurately determine the health status of the target object.

In view of this, the embodiments of the present disclosure provide a human-factor intelligence-based electroencephalogram signal correction method, which can collect a multimodal physiological signal. The multimodal physiological signal includes: the electroencephalogram signal and a target physiological signal other than the electroencephalogram signal. After determining that the target object is subjected to a non-predetermined stimulus based on the target physiological signal, the electroencephalogram signal may be corrected to correct the baseline of the electroencephalogram signal. A similarity between a corrected electroencephalogram signal and an electroencephalogram signal collected without the non-predetermined stimulus is greater than a similarity threshold. It can be seen that the method according to the embodiments of the present disclosure can obtain the electroencephalogram signal for accurately determining the health status of the target object without re-collecting the electroencephalogram signal, thereby improving an acquisition efficiency of the electroencephalogram signal.

The embodiments of the present disclosure provide the human factor intelligence-based electroencephalogram signal correction method. The method is applied to a multimodal physiological signal collection device (hereinafter referred to as the “collection device”). The method includes the following steps.

Step A: a multimodal physiological signal of the target object is collected.

The multimodal physiological signal includes: an electroencephalogram signal and a target physiological signal other than the electroencephalogram signal. The target physiological signal may include at least one of an electrodermal activity (EDA) signal (referred to as an electrodermal signal for short) or a skin temperature (SKT) signal (referred to as a skin temperature signal for short). For example, the target physiological signal may be the EDA signal.

It should be understood that the collection device may include: an electroencephalogram signal collection unit and a target physiological signal collection unit. The target physiological signal collection unit may include at least one of an electrodermal signal collection unit or a skin temperature signal collection unit. The collection device may synchronously collect the electroencephalogram signal and the target physiological signal of the target object (such as a human body) by the electroencephalogram signal collection unit and the target physiological signal collection unit.

Step B: if it is determined that the target object is subjected to a non-predetermined stimulus based on the target physiological signal, the electroencephalogram signal within the multimodal physiological signal is corrected.

In the embodiments of the present disclosure, the collection device may detect, based on the target physiological signal within the multimodal signal, whether the target object is subjected to the non-predetermined stimulus outside an application period of the predetermined stimulus. If the collection device determines that the target object is subjected to non-predetermined stimulus outside the application period of the predetermined stimulus based on the target physiological signal, the electroencephalogram signal may be corrected to correct the baseline of the electroencephalogram signal.

A similarity between a corrected electroencephalogram signal and a reference electroencephalogram signal is greater than a similarity threshold. That is, the corrected electroencephalogram signal is relatively similar to the reference electroencephalogram signal. The similarity between the corrected electroencephalogram signal and the reference electroencephalogram signal may refer to a similarity between a waveform of the corrected electroencephalogram signal and a waveform of the reference electroencephalogram signal. The reference electroencephalogram signal is an electroencephalogram signal collected when the target object is not subject to the non-predetermined stimulus. For example, except for the non-predetermined stimulus, a collection environment of the reference electroencephalogram signal is the same as that of the electroencephalogram signal.

According to the above description, compared with the electroencephalogram signal before correction, the corrected electroencephalogram signal eliminates the interference signal superimposed on the electroencephalogram signal due to the target object being subjected to the non-predetermined stimulus. This ensures high accuracy in determining the obtained health status of the target object based on the corrected electroencephalogram signal.

It should be understood that the third embodiment of the present disclosure is another method example for filtering and artifact removal of the electroencephalogram signal data stream. Compared with the first embodiment, the method according to the third embodiment needs to collect not only the electroencephalogram signal data stream but also the target physiological signal, and uses the target physiological signal to correct an abnormal signal within the electroencephalogram signal data stream. This process can also achieve the purpose of filtering and artifact removal of the electroencephalogram signal data stream.

In the embodiments of the present disclosure, the collection device may perform the step B after collection of the multimodal physiological signal is completed. Alternatively, the collection device may perform the step B during the collection of the multimodal physiological signal. In this way, the acquisition efficiency of the expected electroencephalogram signal can be further improved.

In summary, the embodiments of the present disclosure provide a human-factor intelligence-based electroencephalogram signal correction method. The collection device may collect a multimodal physiological signal. The multimodal physiological signal includes an electroencephalogram signal and a target physiological signal other than the electroencephalogram signal. Moreover, after determining that the target object is subjected to a non-predetermined stimulus based on the target physiological signal, the collection device may correct the electroencephalogram signal. A similarity between the corrected electroencephalogram signal and an electroencephalogram signal collected when the target object is not subject to the non-predetermined stimulus is greater than a similarity threshold. It can be seen that the method according to the embodiments of the present disclosure may obtain the electroencephalogram signal for accurately determining the health status of the target object without re-collecting the electroencephalogram signal of the target object, improving the acquisition efficiency of the electroencephalogram signal.

The embodiments of the present disclosure also provide another human-factor intelligence-based electroencephalogram signal correction method. The method may include the following steps.

Step 1: a multimodal physiological signal of a target object is collected.

The target object may be a human body. The multimodal physiological signal includes an electroencephalogram signal and a target physiological signal other than the electroencephalogram signal. The target physiological signal may be at least one of an EDA signal or a SKT signal. For example, the target physiological signal may be the EDA signal.

It should be understood that the collection device may include an electroencephalogram signal collection unit and a target physiological signal collection unit. The target physiological signal collection unit may include at least one of an electrodermal signal collection unit or a skin temperature signal collection unit. The collection device may synchronously collect the electroencephalogram signal and the target physiological signal of the target object (such as the human body) by the electroencephalogram signal collection unit and the target physiological signal collection unit.

In the embodiments of the present disclosure, the EDA signal is closely related to emotion, an arousal level, attention, and other factors of the target object. In addition, the EDA signal has high stability, is relatively easy to measure, and has high sensitivity. Therefore, the EDA signal is the most effective and sensitive physiological parameter reflecting changes in sympathetic nerve excitability of the individual. In addition, both the EDA signal and the electroencephalogram signal are high-dimensional time-series signals.

Step 2: it is detected whether a mutation has occurred in the target physiological signal with the multimodal physiological signal.

In the embodiments of the present disclosure, after the target object is stimulated, changes occur in the multimodal physiological signal. Since the target physiological signal of the target object changes more significantly than the electroencephalogram signal, the collection device may determine whether the target object is subjected to a non-predetermined stimulus outside an application period of a predetermined stimulus by detecting whether the mutation occurs in the target physiological signal, to ensure high accuracy of determination.

If the collection device determines that no mutation occurs in the target physiological signal outside the application period, the step 3 may be executed. If the collection device determines that a mutation has occurred in the target physiological signal outside the application period, the step 4 may be executed.

It should be understood that if the collection device determines that a difference between a signal value of the target physiological signal collected at a current sampling moment and a signal value of the target physiological signal collected at a previous sampling moment is greater than a second threshold, and the current sampling moment is outside the application period of the predetermined stimulus, it can determine that a mutation has occurred in the target physiological signal.

The second threshold may be pre-stored in the collection device. The predetermined stimulus is an expected stimulus applied to the target object during the collection of the electroencephalogram signal of the target object. The application period of the predetermined stimulus may be pre-stored in the collection device.

Step 3: it is determined that the target object is not subjected to the non-predetermined stimulus.

If the collection device determines that no mutation has occurred in the target physiological signal, it can determine that the target object is not subjected to a non-predetermined stimulus outside the application period of the predetermined stimulus. Correspondingly, there is no need to correct the collected electroencephalogram signal.

It should be understood that in a case where the collection device executes the step 2 during collection of the multimodal physiological signal, if the collection device determines that no mutation has occurred in the currently collected target physiological signal, it can continue to execute the step 2 until the collection of the multimodal physiological signal is completed.

For a case where the collection device executes the step 2 after the collection of multimodal physiological signal is completed, if the collection device determines that no mutation has occurred in the collected target physiological signal, it can determine that the target object is not subjected to a non-predetermined stimulus.

Step 4: it is determined that the target object is subjected to a non-predetermined stimulus.

If the collection device determines that a mutation has occurred in the target physiological signal, it can determine that the target object is subjected to a non-predetermined stimulus outside the application period of the predetermined stimulus, and then proceed to Step 5.

Step 205: the electroencephalogram signal within the multimodal physiological signal is corrected.

The collection device may correct the abnormal signal within the electroencephalogram signal to eliminate the interference signal superimposed on the electroencephalogram signal due to the target object being subjected to the non-predetermined stimulus, thereby achieving the effect of correcting the baseline of the electroencephalogram signal. The abnormal signal refers to the electroencephalogram signal portion collected from the moment when the target object is subject to the non-predetermined stimulus until the target object recovers.

In the embodiments of the present disclosure, the electroencephalogram signal and the target physiological signal are collected synchronously. Therefore, when a mutation occurs in the target physiological signal, the electroencephalogram signal may also change accordingly. Based on this, the collection device may determine a portion of an electroencephalogram signal collected from the occurrence of a mutation in the target physiological signal until the target physiological signal recovers as the abnormal signal within the electroencephalogram signal.

That is, a start sampling moment of the abnormal signal is a moment when the target physiological signal mutates. An end sampling moment is a moment when the target physiological signal recovers. Both the mutation moment and the recovery moment occur outside the application period of the predetermined stimulus. The moment when the target physiological signal mutates is the sampling moment when the signal value first differs from the signal value at the previous sampling moment by more than the second threshold. The recovery moment is the sampling moment, starting from the mutation time, when the signal value first differs from the signal value at the previous sampling moment by less than or equal to the second threshold.

In the embodiments of the present disclosure, the electroencephalogram signal may include multiple signal values. The collection device may correct, based on a chronological order of sampling moments, each of the plurality of abnormal signal values included in the abnormal signal in sequence.

It should be understood that the collection device may correct the abnormal signal within the electroencephalogram signal during the collection of the multimodal physiological signal. That is, the collection device may correct the abnormal signal in real time. In this case, the collection device may correct each abnormal signal value within the abnormal signal in sequence based on an order of sampling moments from earliest to latest.

Alternatively, the collection device may correct the abnormal signal within the electroencephalogram signal after the collection of the multimodal physiological signal is completed. In this case, the collection device may correct each abnormal signal value within the abnormal signal in sequence either in the order of sampling moments from earliest to latest or from latest to earliest.

In the embodiments of the present disclosure, for each abnormal signal value within the abnormal signal, the collection device may correct the abnormal signal value based on a first signal value set corresponding to the abnormal signal value. The first signal value set includes N signal values with consecutive sampling moments. N is an integer greater than 1. A target signal value among the N signal values has a sampling moment adjacent to the sampling moment of the abnormal signal value. The target signal value is the earliest sampled signal value or the latest sampled signal value among the N signal values. That is, sampling moments of the N signal values are all earlier than the sampling moment of the abnormal signal value, or all later than the sampling moment of the abnormal signal value.

A difference between a statistical value of a second signal value set corresponding to the corrected abnormal signal value and a statistical value of the first signal value set is smaller than a first threshold. The second signal value set includes the corrected abnormal value and the N−1 signal values with consecutive sampling moments from the first signal value set. The N−1 signal values include the target signal value. The first threshold may be pre-stored in the collection device.

It should be understood that if the collection device corrects each abnormal signal value in sequence in the order of sampling moments from earliest to latest, the target signal value may be a signal value with the latest sampling moment in the first signal value set. If the collection device corrects each abnormal signal value in sequence in the order of sampling moments from latest to earliest, the target signal value may be a signal value with the earliest sampling moment in the first signal value set.

For example, suppose the first signal value set includes signal values all sampled earlier than a certain abnormal signal value, i.e., all signal values precede the abnormal signal value, and the first signal value set includes: signal value 1, signal value 2, signal value 3, and signal value 4. In this case, the second signal value set may include: signal value 2, signal value 3, signal value 4, and the corrected abnormal signal value.

Suppose the first signal value set includes signal values all sampled later than a certain abnormal signal value, i.e., each signal value following the abnormal signal value, and the first signal value set includes: signal value 1, signal value 2, signal value 3, and signal value 4. In this case, the second signal value set may include: the corrected abnormal signal value, signal value 1, signal value 2, and signal value 3.

It should be understood that the statistical value may include at least one of standard deviation or mean. For example, the statistical value may be the standard deviation. If the statistical value includes both standard deviation and mean, a difference between a standard deviation of the second signal value set and a standard deviation of the first signal value set may be smaller than a first threshold, and a difference between a mean of the second signal value set and a mean of the first signal value set may also be smaller than the first threshold.

In another exemplary embodiment of the present disclosure, the mean may be an arithmetic mean, a geometric mean, or a root mean square.

In another exemplary embodiment of the present disclosure, the N signal values in the first signal value set may be multiple consecutive signal values collected by the collection device within a predetermined time period. The predetermined time period may be pre-stored in the collection device, for example, 5 seconds(s).

It should be understood that a second signal value set corresponding to each corrected abnormal signal value may serve as a first signal value set corresponding to a next abnormal signal value to be corrected. That is, the corrected abnormal signal value may be used to correct the next abnormal signal value.

For example, suppose the abnormal signal includes: abnormal signal value 1 and abnormal signal value 2 arranged in sequence in the order of sampling moments from earliest to latest. A first signal value set corresponding to the abnormal signal value 1 includes: signal value 1, signal value 2, and signal value 3. Assuming that sampling moments of signal value 1 to signal value 3 are all earlier than the sampling moment of the abnormal signal value 1, a second signal value set corresponding to the abnormal signal value 1 may be signal value 2, signal value 3, and the corrected abnormal signal value 1. This second signal value set may serve as a first signal value set corresponding to abnormal signal value 2.

In the embodiments of the present disclosure, the collection device may not only collect the target physiological signal of the target object while collecting the electroencephalogram signal of the target object, but also collect an auxiliary physiological signal of the target object. The auxiliary physiological signals may include one of: an electromyogram (EMG) signal, an electrocardiogram (ECG) signal, an electro-oculogram (EOG) signal, and a photoplethysmography (PPG) signal.

The EMG signal is a bioelectrical current generated by contraction of a surface muscle of the human body. A result of the ECG signal is typically displayed in a waveform. The EMG signal basically includes P wave, QRS complexes, and T wave. The P wave represents atrial contraction. The QRS complex represents ventricular contraction. The T wave represents ventricular relaxation.

The photoplethysmography (PPG) signal is detected by photoelectric technology and may reflect changes in a blood volume in a peripheral blood vessel caused by cardiac activity. When light of a determined wavelength irradiates a skin surface, contraction and expansion of a blood vessel with each heartbeat may affect transmission or reflection of light. When the light passes through a skin tissue and is reflected back to a photoelectric receiver, light intensity may be attenuated to a certain extent. While the absorption of light by other tissues (such as muscles, bones, and veins) remains relative constant, absorption of light by arteries varies due to pulsation of blood. Based on this, when an optical signal is converted into an electrical signal, an obtained electrical signal may be divided into a direct current signal and an alternating current signal. The alternating current signal may reflect a characteristic of blood flow. It should be understood that a choice of transmission or reflection depends on a test site. Typically, light transmission is used for fingers, while reflection is mostly used for wrists.

According to the above description, the collection device may monitor changes in multiple types of physiological signals simultaneously. That is, the collection device is a multimodal apparatus capable of collecting multiple types of physiological signals simultaneously. This enables the capture of physiological data of the target object can be from multiple different perspectives, allowing for relatively comprehensive monitoring of the health status of the target object. Consequently, this ensures higher accuracy and reliability of the analyzed health status of the target object. The health status may include physical health status and mental health status. The mental health status may be reflected by emotion, stress, and other factors.

In the embodiments of the present disclosure, the collection device can not only collect multiple types of physiological signals synchronously, but also perform feature fusion processing on the collected physiological signal to obtain a fused signal. After that, the collection device can process the fused signal by a classification model to obtain health status of the target object. The classification model may be pre-stored in the collection device.

It should be understood that the collection device may perform alignment and feature fusion processing on multiple types of physiological signals by a hardware circuit in the collection device and an embedded algorithm programmed in the hardware circuit to generate the fused signal.

Before performing feature fusion processing on multiple types of physiological signals, the collection device can perform feature extraction from the collected physiological signal. For example, the collection device may extract at least one of a time-domain feature (such as mean and variance) or a frequency-domain feature of the physiological signal. For example, the collection device may extract both the time-domain feature and the frequency-domain feature of the physiological signal. Feature extraction is a process of converting an original physiological signal into a feature vector.

It should be understood that the collection device can process the physiological signal using a time-domain feature extraction method (or a frequency-domain feature extraction method) to extract the time-domain feature (or the frequency-domain feature) of the physiological signal. In another exemplary embodiment of the present disclosure, the time-domain feature extraction method may include wavelet transform and short-time Fourier transform. The frequency feature extraction method may include discrete Fourier transform and power spectral density.

In another exemplary embodiment of the present disclosure, the classification model may be obtained by training multiple pieces of sample data using a machine learning algorithm. The machine learning algorithm may be one of support vector machine, artificial neural network, and decision tree. Each piece of sample data may include a sample physiological signal and a health status corresponding to the sample physiological signal.

It should be understood that the collection device may also determine blood oxygen saturation (pulse oximeter oxygen saturation, SpO₂), heart rate (HR), and blood pressure based on the PPG signal. In addition, the collection device may determine the blood pressure based on the ECG signal.

It should be understood that a sequence of steps in the human-factor intelligence-based electroencephalogram signal correction method according to the embodiments of the present disclosure may be adjusted as appropriate. The steps may be added or removed as desired. Any method variations readily conceivable by those skilled in the art within the technical scope disclosed in the present disclosure shall fall within the protection scope of the present disclosure, and details thereof will be omitted here.

In summary, the embodiments of the present disclosure provide a human-factor intelligence-based electroencephalogram signal correction method. The collection device may collect a multimodal physiological signal. The multimodal physiological signal includes an electroencephalogram signal and a target physiological signal other than the electroencephalogram signal. Moreover, after determining that the target object is subjected to the non-predetermined stimulus based on the target physiological signal, the collection device may correct the electroencephalogram signal. A similarity between the corrected electroencephalogram signal and an electroencephalogram signal collected when the target object is not subject to a non-predetermined stimulus is greater than a similarity threshold. It can be seen that the method according to the embodiments of the present disclosure can obtain the electroencephalogram signal for accurately determining health status of the target object without re-collecting the electroencephalogram signal of the target object, thereby improving the acquisition efficiency of the electroencephalogram signal.

The embodiments of the present disclosure provide the human-factor intelligence-based electroencephalogram signal correction apparatus. The apparatus may be configured to perform the human-factor intelligence-based electroencephalogram signal correction method according to the above method embodiments. The apparatus includes: a collection module configured to collect a multimodal physiological signal of the target object, where the multimodal physiological signal includes an electroencephalogram signal and a target physiological signal other than the electroencephalogram signal; and a correction module configured to correct the electroencephalogram signal if it is determined based on the target physiological signal that the target object is subjected to a non-predetermined stimulus.

The similarity between the corrected electroencephalogram signal and a reference electroencephalogram signal is greater than a similarity threshold. The reference electroencephalogram signal is an electroencephalogram signal collected when the target object is not subjected to a non-predetermined stimulus.

In one implement, the correction module may be configured to correct an abnormal signal within the electroencephalogram signal.

The abnormal signal refers to a portion of the electroencephalogram signal collected during a period from the target object being subject to the non-predetermined stimulus until the target object recovers.

In one implement, the electroencephalogram signal includes a plurality of signal values. The correction module may be configured to correct, based on the chronological order of sampling moments, each abnormal signal value included in the abnormal signal in sequence.

In one implement, the correction module may be configured to, for each abnormal signal value within the abnormal signal, correct the abnormal signal value based on a first signal value set corresponding to the abnormal signal value.

The first signal value set includes N signal values with consecutive sampling moments. A target signal value among the N signal values has a sampling moment adjacent to the sampling moment of the abnormal signal value. The target signal value is the earliest sampled signal value or the latest sampled signal value among the N signal values.

A difference between a statistical value of a second signal value set corresponding to the corrected abnormal signal value and a statistical value of the first signal value set is smaller than a first threshold. The second signal value set includes the corrected abnormal value and N−1 signal values with consecutive sampling moments from the first signal value set. The N−1 signal values include the target signal value.

In one implement, the statistical value includes at least one of standard deviation or mean.

In one implement, the apparatus may further include: a first determination module configured to determine that the target object is subject to the non-predetermined stimulus in response to determining that a mutation occurs in the target physiological signal.

In one implement, the apparatus may further include: a first determination module configured to determine that a mutation occurs in the target physiological signal in response to a difference between a signal value of the target physiological signal collected at a current sampling moment and a signal value of the target physiological signal collected at a previous sampling moment being greater than a second threshold, and the current sampling moment being outside an application period of a predetermined stimulus

In one implement, the apparatus may further include: a third determination module configured to determine a portion of the electroencephalogram signal collected during a period from the occurrence of a mutation in the target physiological signal until the target physiological signal recovers as the abnormal signal.

In one implement, the target physiological signal is at least one of a skin temperature signal or an electrodermal activity signal.

In summary, the embodiments of the present disclosure provide a human-factor intelligence-based electroencephalogram signal correction apparatus. The apparatus may collect a multimodal physiological signal. The multimodal physiological signal includes an electroencephalogram signal and a target physiological signal s other than the electroencephalogram signal. Moreover, after determining that the target object is subjected to the non-predetermined stimulus based on the target physiological signal, the collection device may correct the electroencephalogram signal. The similarity between the corrected electroencephalogram signal and an electroencephalogram signal collected when the target object is not subject to a non-predetermined stimulus is greater than a similarity threshold. It can be seen that the apparatus can obtain the electroencephalogram signal for accurately determining health status of the target object without re-collecting the electroencephalogram signal of the target object, thereby improving the acquisition efficiency of the electroencephalogram signal.

FIG. 8 is a schematic diagram of a structure of an electronic device according to an embodiment of the present disclosure. The electronic device may be the electronic device in the second embodiment or the collection device in the third embodiment. As illustrated in FIG. 8, the electronic device 400 includes a processor 401 and a memory 403. The processor 401 is connected to the memory 403, for example, via a bus 402. In one implement, the electronic device 400 may further include a transceiver 404. It should be noted that in practical applications, a number of the transceiver 404 is not limited to one. A structure of the collection device 400 does not constitute a limitation on the embodiments of the present disclosure.

The processor 401 may be a Central Processing Unit (CPU), a general-purpose processor, a Digital Signal Processor (DSP), an Application Specific Integrated Circuit (ASIC), a Field Programmable Gate Array (FPGA), or other programmable logic devices, transistor logic devices, hardware components, or any combination thereof. The processor may implement or execute various exemplary logical blocks, modules, and circuits described in conjunction with the contents of the present disclosure. The processor 401 may also be a combination of computing functions, such as a combination of one or more microprocessors, or a combination of a DSP and a microprocessor.

The bus 402 may include a path for transmitting information between the above components. The bus 402 may be a Peripheral Component Interconnect (PCI) bus, an Extended Industry Standard Architecture (EISA) bus, or the like. The bus 402 may be classified into an address bus, a data bus, a control bus, etc. For ease of representation, only one thick line is used in FIG. 8, but this does not mean that there is only one bus or one type of bus.

The memory 403 is configured to store a computer program corresponding to the human factor intelligence-based electroencephalogram signal correction method in the embodiments of the present disclosure. The computer program is controlled and executed by the processor 401. The processor 401 is configured to execute the computer program stored in the memory 403 to implement the contents shown in the first method embodiment, the second method embodiment, or the third method embodiment.

The embodiments of the present disclosure further provide a computer-readable storage medium having a computer program stored thereon. The computer program, when executed by a processor, implements the method for real-time artifact processing and feature extraction according to the first embodiment, the human-factor intelligence-based electroencephalogram signal processing method according to the second embodiment, or the human-factor intelligence-based electroencephalogram signal correction method according to the third embodiment.

What is claimed is:

1. A method for real-time artifact processing and feature extraction of an electroencephalogram signal, wherein the method comprises:
receiving in real time an electroencephalogram signal data stream collected by an electroencephalographic device;
segmenting the electroencephalogram signal data stream in real time via a sliding window approach;
obtaining parameter information of the electroencephalographic device, and performing, based on the parameter information, adaptive filtering and artifact removal on a segment of the electroencephalogram signal data stream, the parameter information comprising a number of channels and a sampling rate; and
matching a feature extraction strategy for one or more pre-selected output indicators, and extracting in real time, based on the matched feature extraction strategy, a feature value conforming to the output indicators from the filtered and artifact-removed segment of the electroencephalogram signal data stream from perspectives comprising time domain analysis, frequency domain analysis, time-frequency domain analysis, and/or nonlinear analysis.

2. The method according to claim 1, further comprising, prior to receiving the electroencephalogram signal data stream and performing artifact processing and feature extraction:
obtaining device information of the electroencephalographic device that provides the electroencephalogram signal data stream, the device information comprising the number of channels and the sampling rate; and/or
obtaining a user configuration for real-time artifact processing and feature extraction, the user configuration comprising a time window size of a sliding window, a filtering strategy, an artifact removal strategy, a feature value output indicator, and a selected protocol supporting feature value transmission.

3. The method according to claim 1, wherein a protocol supporting transmission of each of the electroencephalogram signal data stream collected by the electroencephalographic device and the extracted feature value comprises one or more of TCP protocol, UDP protocol, wireless Bluetooth protocol, MQTT protocol, RS-232/RS-485 protocol, and LSL protocol; and/or
the filtering comprises one or more of low-pass filtering, high-pass filtering, band-pass filtering, and notch filtering; and
the artifact removal comprises one or more of independent component analysis, global field power analysis, blind source signal separation, and SVM-based artifact removal.

4. The method according to claim 3, wherein:
in a scenario of identifying an anxiety state based on the electroencephalogram signal, an energy value of a frequency band, an energy value of θ frequency band, and an energy value of γ frequency band are used as the output indicators, and the artifact removal comprises the independent component analysis and the global field power analysis; and
in a scenario of prosthesis control based on the electroencephalogram signal, a feature value corresponding to event-related synchronization and desynchronization output indicators of μ rhythm and β rhythm is extracted in real time from the filtered and artifact-removed segment of the electroencephalogram signal data stream from the perspective of time-frequency domain analysis based on the matched feature extraction strategy.

5. The method according to claim 1, wherein said extracting in real time, based on the matched feature extraction strategy, the feature value conforming to the output indicators from the filtered and artifact removed segment of the electroencephalogram signal data stream from the perspectives comprising time domain analysis, frequency domain analysis, time-frequency domain analysis, and/or nonlinear analysis comprises:
from the perspective of time domain analysis, extracting in real time, based on a statistical algorithm or an Hjorth algorithm, one or more of a mean value, a variance, a standard deviation, a kurtosis, a skewness, and an autocorrelation coefficient from the filtered and artifact removed segment of electroencephalogram signal data stream;
from the perspective of frequency domain analysis, extracting in real time an energy value and/or a power value from the filtered and artifact removed segment of the electroencephalogram signal data stream based on any one of fast Fourier transform algorithm, periodogram method, Welch method, multi-window method, and autoregressive model;
from the perspective of time-frequency domain analysis, extracting in real time, based on short-time Fourier transform or continuous wavelet transform, the feature value from the filtered and artifact-removed segment of the electroencephalogram signal data stream; and
from the perspective of nonlinear analysis, extracting in real time, based on recursive variable analysis and complexity, one or more of Shannon entropy, approximate entropy, sample entropy, and permutation entropy from the filtered and artifact-removed segment of the electroencephalogram signal data stream.

6. The method according to claim 1, wherein said performing, based on the parameter information, adaptive filtering and artifact removal on the segment of the electroencephalogram signal data stream comprises:
when a number of channels of the electroencephalogram signal collected by the electroencephalographic device is smaller than a predetermined threshold, performing, by using a pre-trained neural network model, adaptive filtering and artifact removal on the segment of the electroencephalogram signal data stream, wherein the neural network model is obtained through supervised learning and training using a large-scale dataset of electroencephalogram signals containing different types of artifacts and/or having different signal-to-noise ratios.

7. The method according to claim 1, wherein said the step of extracting in real time, based on the matched feature extraction strategy, the feature value conforming to the output indicators from the filtered and artifact-removed segment of the electroencephalogram signal data stream from perspectives comprising time domain analysis, frequency domain analysis, time-frequency domain analysis, and/or nonlinear analysis is performed based on a convolutional neural network model, wherein the convolutional neural network model comprises a first feature extraction module, a second feature extraction module, and a third feature extraction module that are connected in sequence, each of the first feature extraction module, the second feature extraction module, and the third feature extraction module comprising a temporal convolution kernel for extracting temporal feature information, and the second feature extraction module further comprising a spatial convolution kernel for extracting spatial feature information, wherein the method comprises:
inputting a to-be-processed electroencephalogram signal into the first feature extraction module, the second feature extraction module, and the third feature extraction module that are connected in sequence, to obtain temporal feature information and spatial feature information of the electroencephalogram signal.

8. The method according to claim 7, wherein the convolutional neural network model further comprises a classification module, and the method further comprises:
inputting the temporal feature information and the spatial feature information of the to-be-processed electroencephalogram signal into the classification module for classification processing to obtain a classification processing result of the electroencephalogram signal.

9. The method according to claim 8, wherein:
the first feature extraction module comprises at least one depthwise separable convolutional layer, each of the at least one depthwise separable convolutional layer comprising a first temporal convolution kernel for extracting temporal feature information; and
said inputting the to-be-processed electroencephalogram signal into the first feature extraction module, the second feature extraction module, and the third feature extraction module that are connected in sequence comprises:
inputting the to-be-processed electroencephalogram signal into the first feature extraction module to obtain a first feature map output from the first feature extraction module, the first feature map comprising temporal feature information extracted by the first temporal convolution kernel.

10. The method according to claim 9, wherein:
the second feature extraction module comprises at least one standard convolutional layer and at least one residual block that are connected in sequence, the at least one standard convolutional layer comprising the spatial convolution kernel for extracting the spatial feature information, and the at least one residual block comprising a second temporal convolution kernel for extracting the temporal feature information; and
said inputting the to-be-processed electroencephalogram signal into the first feature extraction module, the second feature extraction module, and the third feature extraction module that are connected in sequence comprises:
inputting the first feature map into the second feature extraction module to obtain a second feature map output from the second feature extraction module, the second feature map comprising the spatial feature information extracted by the spatial convolution kernel and a first optimized temporal feature information extracted by the second temporal convolution kernel.

11. The method according to claim 10, wherein the second feature extraction module comprises a first residual block and a second residual block that are connected in sequence, wherein:
the first residual block comprises a first convolutional layer, a second convolutional layer, and a first skip connection that are connected in sequence, the first skip connection is configured to add input information of the first convolutional layer to output information of the second convolutional layer to serve as output information of the first residual block; and
the second residual block comprises a third convolutional layer, a fourth convolutional layer, and a second skip connection that are connected in sequence, the second skip connection is configured to add input information of the third convolutional layer to output information of the fourth convolutional layer to serve as output information of the second residual block,
wherein the first convolutional layer, the second convolutional layer, the third convolutional layer, and the fourth convolutional layer each comprises the second temporal convolution kernel.

12. The method according to claim 11, wherein:
the third feature extraction module comprises at least one mixed dilated convolutional layer, and each of the at least one mixed dilated convolutional layer comprises a third temporal convolution kernel for extracting temporal feature information; and
said inputting the to-be-processed electroencephalogram signal into the first feature extraction module, the second feature extraction module, and the third feature extraction module that are connected in sequence comprises:
inputting the second feature map into the third feature extraction module to obtain a third feature map output from the third feature extraction module, the third feature map comprising the spatial feature information extracted by the spatial convolution kernel and a second optimized temporal feature information extracted by the third temporal convolution kernel.

13. The method according to claim 12, wherein the third feature extraction module in comprises a first mixed dilated convolutional layer and a second mixed dilated convolutional layer that are connected in sequence, and the third temporal convolution kernel has a size of (1, c), where c is a positive integer greater than 1.

14. The method according to claim 1, further comprising:
collecting a target physiological signal other than the electroencephalogram signal data stream; and
correcting the electroencephalogram signal data stream in response to determining, based on the target physiological signal, that a target object is subject to a non-predetermined stimulus,
wherein a similarity between the corrected electroencephalogram signal data stream and a reference electroencephalogram signal is greater than a similarity threshold, the reference electroencephalogram signal being an electroencephalogram signal collected when the target object is not subject to the non-predetermined stimulus.

15. The method according to claim 14, wherein said correcting the electroencephalogram signal data stream comprises:
correcting an abnormal signal within the electroencephalogram signal data stream,
wherein the abnormal signal is a portion of the electroencephalogram signal collected during a period from the target object being subject to the non-predetermined stimulus until the target object recovers.

16. The method according to claim 15, wherein the electroencephalogram signal data stream comprises a plurality of signal values, and said correcting the abnormal signal comprises:
correcting abnormal signal values comprised in the abnormal signal in sequence based on a chronological order of sampling moments.

17. The method according to claim 16, wherein said correcting the abnormal signal values comprised in the abnormal signal in sequence comprises:
for each of the abnormal signal values within the abnormal signal, correcting the abnormal signal value based on a first signal value set corresponding to the abnormal signal value, wherein:
the first signal value set comprises N signal values with consecutive sampling moments, a target signal value among the N signal values has a sampling moment adjacent to a sampling moment of the abnormal signal value, and the target signal value is the earliest sampled signal value or the latest sampled signal value among the N signal values; and
a difference between a statistical value of a second signal value set corresponding to the corrected abnormal signal value and a statistical value of the first signal value set is smaller than a first threshold, wherein the second signal value set comprises the corrected abnormal value and N−1 signal values with consecutive sampling moments from the first signal value set, and the N−1 signal values comprise the target signal value.

18. The method according to claim 14, further comprising:
determining that the target object is subject to the non-predetermined stimulus in response to determining that a mutation has occurred in the target physiological signal.

19. The method according to claim 18, further comprising:
determining that the mutation has occurred in the target physiological signal in response to a difference between a signal value of the target physiological signal collected at a current sampling moment and a signal value of the target physiological signal collected at a previous sampling moment being greater than a second threshold and the current sampling moment being outside an application period of a predetermined stimulus.

20. An apparatus for real-time artifact processing and feature extraction of an electroencephalogram signal, the apparatus comprising:
a memory having a computer instruction stored therein; and
a processor configured to execute the computer instruction stored in the memory, wherein the apparatus is configured to, when the computer instruction is executed by the processor, implement a method for real-time artifact processing and feature extraction of an electroencephalogram signal, wherein the method comprises:
receiving in real time an electroencephalogram signal data stream collected by an electroencephalographic device;
segmenting the electroencephalogram signal data stream in real time via a sliding window approach;
obtaining parameter information of the electroencephalographic device, and performing, based on the parameter information, adaptive filtering and artifact removal on a segment of the electroencephalogram signal data stream, the parameter information comprising a number of channels and a sampling rate; and
matching a feature extraction strategy for one or more pre-selected output indicators, and extracting in real time, based on the matched feature extraction strategy, a feature value conforming to the output indicators from the filtered and artifact-removed segment of the electroencephalogram signal data stream from perspectives comprising time domain analysis, frequency domain analysis, time-frequency domain analysis, and/or nonlinear analysis.
